(19) United States
(12) Patent Application Publication
(10) Pub. No.: US 2005/0053974 A1
(43) Pub. Date: Mar. 10, 2005

(54) Apparatus and methods for surface plasmon-coupled directional emission

(75) Inventors: Joseph R. Lakowicz, Ellicott City, MD (US); Joanna Malicka, Baltimore, MD (US); Ignacy Gryczynski, Baltimore, MD (US); Zygmunt Gryczynski, Ellicot City, MD (US)

Correspondence Address: BURNS DOANE SWECKER & MATHIS L L P, POST OFFICE BOX 1404, ALEXANDRIA, VA, 22313-1404, US

(73) Assignee: University of Maryland, Baltimore, MD (US)

(21) Appl. No.: 10/849,465

(22) Filed: May 20, 2004

Related U.S. Application Data
(60) Provisional application No. 60/471,918, filed on May 20, 2003.

(51) Int. Cl. C12Q 1/68

(52) U.S. Cl. 435/6

(57) ABSTRACT

Methods and apparatus for fluorescence detection which can increase sensitivity by as much as 20 to 1000-fold are described. This method can preferably also decrease the contribution of sample autofluorescence to the detected signal. The method uses coupling of excited fluorophores with the surface plasmon resonance present in thin conductive films, for example silver, gold, aluminum, copper, or the like. The phenomenon of surface plasmon-coupled emission (SPCE) occurs for fluorophores in a volume adjacent to the conductive layer. This interaction is independent of the mode of excitation, that is, does not require evanescent wave or surface-plasmon excitation. However, such modes of excitation can be advantageous. SPCE can occur over a narrow angular distribution, converting normally isotropic emission into easily collected directional emission. In preferred embodiments, up to 50% of the emission from unoriented samples can be collected, usually much more than typical fluorescence collection efficiencies, which can be 1% or less. Examples are presented showing how simple optical configurations can be used in diagnostics, sensing, or biotechnology applications. Surface plasmon-coupled emission is likely to find widespread applications throughout the biosciences.

### RELATED APPLICATIONS

This application claims benefit of U.S. Provisional Application No. 60/471,918, filed May 20, 2003, which is incorporated herein in its entirety.

### GOVERNMENT FUNDING

This work was supported at least in part by the NIH National Center for Research Resource, RR-08119, HG-002655, EB-000682 and EB-00981. The United States Government may have certain rights in the invention.

### BACKGROUND OF THE INVENTION

1. Field of the Invention

The apparatus and methods described herein relate to improvements in fluorescence spectroscopy, which is useful for example in the filed of analytical biochemistry, genomics, proteomics, and cellular and tissue imaging.

2. Description of the Related Art

Fluorescence detection is used for numerous assays in the biological sciences, biotechnology and medical diagnostics. Fluorescence is a highly sensitive method, but there is always a need for increased sensitivity to detect smaller numbers of target molecules. Numerous methods have been developed to increase sensitivity. These methods include amplified assays such as Elisa (Gosling, J. P., 1990, A decade of development in immunoassay methodology, Cell, 36: 1408-1427) and PCR (Walker, N.J., 2002, A technique whose time has come, Science, 296;557-559), probes with multiple fluorophores such as the phycobiliproteins (White, J. C., and Stryer, L., 1987, Photostability studies of phycobiliprotein fluorescent labels, Anal. Biochem. 161: 442-452; Kronick, M. N., 1986, The use of phycobiliproteins as fluorescent labels in immunoassays, J. Immunological Methods 92: 1-13), long wavelength probes (Daehne, S., Resch-Genger, U., and Wolfbeis, O. S. (Eds.), 1998, Near-Infrared Dyes for High Technology Applications, Kluwer Academic Publishers, New York, 458 pp.; Casay, G. A., Shealy, D. B., and Patonay, G., 1994, Near-infrared fluorescence probes, in Topics in Fluorescence Spectroscopy, Vol. 4: Probe Design and Chemical Sensing, (Lakowicz, J. R., Ed), Plenum Press, New York, pp. 183-222) and/or gated detection to decrease the background emission (Diamandis, E. P., 1988, Immunoassays with time-resolved fluorescence spectroscopy: Principles and applications, Clin. Biochem. 21: 139-150; Lövgren, T., and Pettersson, K., 1990, “Time-resolved fluoroimmunoassay, advantages and limitations,” in Luminescence Immunoassay and Molecular Applications, K. Van Dyke and R. Van Dyke (Eds.), CRC Press, New York, pp. 234-250). Several fundamental factors limit the sensitivity of fluorescence methods, typically photodestruction of the fluorophores and the extent of background fluorescence.

Conventional fluorescence detection relies on the emission of fluorophores under free-space conditions, that is, emission into a transparent non-absorbing medium typical of biological samples. In such samples the fluorophores emit mostly isotropically in all directions with a radiative decay rate (Γ) given approximately by the Strickler and Berg equation (Strickler, S. J., and Berg, R. A., 1962, Relationship between absorption intensity and fluorescence lifetimes of molecules, J. Chem. Phys., 37: 814-822). In some conditions the radiative decay rate can be changed. One well known example is the effect of refractive index on fluorescence decay times as exemplified by the Strickler and Berg equation (Strickler, S. J., and Berg, R. A., supra) showing the radiative decay rate (Γ) of an oscillating dipole increases in proportion to n², where n is the refractive index of the medium.

The effects of refractive index on fluorescence are modest and are seldom used in fluorescence experiments (Lieberherr, M., Fattinger, Ch., and Lukosz, W., 1987, Optical-environment-dependent effects on the fluorescence of submonomolecular dye layers on interfaces, Surface Science 189/190: 954-959; Toptygin, D., Savtchenko, R. S., Meadow, N. D., Roseman, S., and Brand, L., 2002, Effect of the solvent refractive index on the excited-state lifetime of a single-tryptophan residue in a protein, J. Phys. Chem. B., 106: 3724-3734; Lukosz, W., and Kunz, R. E., 1979, Changes in fluorescence lifetimes induced by variation of the radiating molecules optical environment, Optics Commun., 31(1): 42-46). However, one way to change radiative decay rates and spatial distribution of radiated energy involves metallic surfaces or particles, typically silver and gold. Examples of the effects of metals on fluorescence include the oscillations of lifetimes with distance in front of a mirror (Dhexhage, K. H., 1974, Interaction of light with monomolecular dye lasers, in Progress in Optics (Wolfe, E., Ed), North Holland, Amsterdam, pp. 161-232; Amos, R. M., and Barnes, W. L., 1997, Modification of the spontaneous emission rate of Eu3+ ions close to a thin metal mirror, Phys. Rev. B., 55(11): 7249-7254) and the decreased emission rate of fluorophores between closely spaced mirrors. (Hinds, E. A., 1991, Cavity quantum electrodynamics, Adv. At. Mol. Opt. Phys., 28: 237-289; Haroche, S., and Kleppner, D., 1989, Cavity quantum electrodynamics, Physics Today, 24-30; Haroche, S., and Raimond, J.-M., 1993, Cavity quantum electrodynamics, Scientific American, 54-62). The effects in front of a mirror are modest, typically 30%, and fewer results are available for fluorophores between mirrors with nanometer scale distances.

Another approach for modification of the emissive properties of fluorophores involves use of conducting metallic silver particles and colloids. Proximity of fluorophores to silver particles can result in increased intensities, quantum yields and photostability (Lakowicz, J. R., 2001, Radiative decay engineering: Biophysical and biomedical applications, Appl. Biochem., 2981: 1-24; Lakowicz, J. R., Shen, Y., D'Auria, S., Malicka, J., Gryczynski, Z. And Gryczynski, I., 2002, Radiative decay engineering 2: Effects of silver island films on fluorescence intensity, lifetimes and resonance energy transfer, Anal. Biochem., 301: 261-277; Lakowicz, J. R., Gryczynski, I., Shen, Y., Malicka, J., and Gryczynski, Z., 2001, Intensified fluorescence, Photonics Spectra, 96-104; Lakowicz, J. R., Malicka, J., Gryczynski, I., 2003, Silver particles enhance the emission of fluorescent DNA oligomers, BioTechniques, 34: 62-68.; Malicka, J., Gryczynski, I., Fang, J., and Lakowicz, J. R., 2003, Fluorescence spectral properties of cyanine dye-labeled DNA oligomers on surfaces coated with silver particles, Anal. Biochem., 317: 136-146). These effects can be accompanied by dramatically decreased lifetimes, indicating a substantial increase in the radiative decay rates. Release of fluorophores self-quenching can also (Lakowicz, J. R., Malicka, J., D'Auria, S., and Gryczynski, I., 2003, Release of the Self-Quenching of Fluorescence Near Silver Metallic Surfaces, Anal. Biochem., 320: 13-20; Malicka, J., Gryczynski, I., and Lakowicz, J. R., 2003, Enhanced Emission of Highly Labeled DNA Oligomers Near Silver Metallic Surfaces, Anal. Chem., 75: 4408-4414) and also enhanced emission near fractal and light-deposited silver structures can occur (Parfenov, A., Gryczynski, I., Malicka, J., Geddes, C. D., and Lakowicz, J. R., 2003, Enhanced Fluorescence from Fluorophores on Fractal Silver Surfaces. J. Phys. Chem. B., 107: 8829-8833.; Geddes, C. D., Parfenov, A., and Lakowicz, J. R., 2003, Photodeposition of Silver Can Result In Metal-Enhanced Fluorescence, Applied Spec., 57: 526-531). These results illustrate the useful potential of modifying fluorescence using metals, e.g. for increased sensitivity.

One approach for increasing sensitivity is to increase the fraction of the total emission collected by the instrument. Fluorescence in solution is isotropic and it is difficult to collect more than a small fraction of the emitted photons. The importance of light collection efficiency can be seen by consideration of the requirements for single molecule detection (Ambrose, W. P., Goodwin, P. M., Jett, J. H., Van Orden, A., Wemer, J. H., and Keller, R. A., 1999, Single molecule fluorescence spectroscopy at ambient temperature, Chem. Rev., 99: 2929-2956; Soper, S. A., Nutter, H. L., Keller, R. A., Davis, L. M., and Shera, E. B., 1993, The photophysical constants of several fluorescent dyes pertaining to ultrasensitive fluorescence spectroscopy, Photochem. and Photobiol., 57(6): 972-977). A typical fluorophore can undergo a finite number of excitation-relaxation cycles prior to photochemical destruction. For photostable molecules, such as tetramethylrhodamine, photodestruction occurs after about 105 cycles. However, the number of photons detectable from a single fluorophore is typically much smaller, near 103 photons. This decrease is due in part to the isotropic distribution of fluorescence, which makes it difficult to capture more than a small fraction of the total emission. According to commentators, even efficient detection systems capture only about 1% of the total emission, and typically less (Van Orden, A., Machara, N. P., Goodwin, P. M., and Keller, R. A., 1998, Single-molecule identification in flowing sample streams by fluorescence burst size and intraburst fluorescence decay rate, Anal. Chem., 70(7): 1444-1451). Higher collection efficiencies near 10% are possible (Schmidt, Th., Schutz, G. J., Baumgartner, W., Gruber, H. J., and Schindler, H., 1995, Characterization of photophysics and mobility of single molecules in a fluid lipid membrane, J. Phys. Chem., 99: 17662-17668; Köhn, F., Hofkens, J., Gronheid, R., Van der Auweraer, M., and DeSchryver, F. C., 2002, Parameters influencing the on- and off-times in the fluorescence intensity traces of single cyanine dye molecules, J. Phys. Chem. A., 106: 4808-4814), but at the expense of complex optics.

### SUMMARY OF THE INVENTION

An apparatus for detecting fluorescence in biochemical assays using surface plasmon-coupled emission is described. An exemplary apparatus can include a first layer of conductive material, for example a metal such as silver, gold, aluminum, copper, or the like, arranged on a first medium, the first medium having a first index of refraction and being a solid medium. The first layer of conductive material is preferably situated at an interface between said first medium and a second medium, the second medium having a second index of refraction different from the first index of refraction. The apparatus can include a second layer comprising functional molecules disposed on the first layer, the functional molecules comprising at least one of nucleic acid molecules and polypeptide molecules. Further, the functional molecules can include one or more types of fluorophores and/or the functional molecules can be capable of binding analyte molecules comprising one or more types of fluorophores. The apparatus preferably includes an excitation source capable of exciting fluorophores positioned adjacent to the first layer and a light detector arranged to selectively detect emitted light that is generated by excited fluorophores. The detector is preferentially arranged to collect emitted light over a predetermined angular range relative to a surface of the first layer. The detected emitted light preferentially emanates from the first layer at the surface plasmon angle, relative to a surface of said first layer, for an emission wavelength of the excited fluorophores. The emitted light preferably passing through the first medium before being detected by the detector. Thus, the predetermined angular range of the detector preferably comprises the surface plasmon angle for the emission wavelength of the excited fluorophores.

Methods for detecting fluorescence in biochemical assays using surface plasmon-coupled emission. In an exemplary embodiment, the method can include: arranging an assay device proximate to a light detector, the assay device comprising a first layer of conductive material arranged on a first medium, the first medium having a first index of refraction and being a solid medium, said first layer of conductive material being situated at an interface between said first medium and a second medium, the second medium having a second index of refraction different from the first index of refraction. Such an assay device preferably also includes a second layer comprising functional molecules disposed on the first layer, the functional molecules comprising at least one of nucleic acid molecules and polypeptide molecules, the functional molecules being capable of binding analyte molecules comprising one or more types of fluorophores. In preferred embodiments, fluorophores are caused to be adjacent to said first layer of said assay device. At least some of the fluorophores are excited with an excitation source. Emitted light that is generated by excited fluorophores is detected with a detector, the emitted light having an emission wavelength of the fluorophores. The emitted light preferably emanates from the first layer of conductive material at the surface plasmon angle corresponding to the emission wavelength relative to a surface of said first layer and passes through said first medium before being detected by the detector.

An alternative exemplary apparatus for observing surface plasmon-coupled emission is also described. In this exemplary embodiment, the apparatus can include an optical fiber having a first index of refraction and having a surface portion coated with a first layer of conductive material, the first layer of conductive material being situated at an interface between the optical fiber and a medium, the medium having a second index of refraction different from the first index of refraction. Such an apparatus can further include a second layer comprising functional molecules disposed on the first layer, the functional molecules comprising at least one of nucleic acid molecules and polypeptide molecules, the functional molecules comprising one or more types of fluorophores and/or the functional molecules can be capable of binding to analyte molecules comprising one or more types of fluorophores. Such an apparatus can further include an excitation source capable of exciting fluorophores positioned adjacent to the first layer and a light detector optically coupled to the optical fiber and arranged to collect emitted light generated by excited fluorophores, where the emitted light passes through the optical fiber to the detector, the emitted light having an emission wavelength of the fluorophores.

Accordingly, an alternative exemplary method for observing surface plasmon-coupled emission can include optically coupling an optical fiber to a light detector, the optical fiber having a first index of refraction and having a surface portion coated with a first layer of conductive material, the first layer of conductive material being situated at an interface between the optical fiber and a medium, the medium having a second index of refraction different from the first index of refraction, the optical fiber further having a second layer comprising functional molecules disposed on the first layer, the functional molecules comprising at least one of nucleic acid molecules and polypeptide molecules, the functional molecules comprising one or more types of fluorophores and/or being capable of binding to analyte molecules comprising one or more types of fluorophores; causing fluorophores to be adjacent to said first layer of conductive material; exciting at least some of said fluorophores adjacent to said first layer with an excitation source; and detecting light generated by excited fluorophores with the detector, the emitted light passing through the optical fiber to the detector, the emitted light having an emission wavelength of the fluorophores.

In an alternative embodiment an exemplary apparatus for observing surface plasmon-coupled emission can include a layer of conductive material arranged on a first medium, the first medium having a first index of refraction and being a solid medium, the layer of conductive material being situated at an interface between the first medium and a second medium, the second medium having a second index of refraction different from the first index of refraction, the layer of conductive material comprising a patterned structure. Such an apparatus preferably includes one or more types of fluorophores positioned adjacent to said layer of conductive material. Further, such an apparatus preferably includes an excitation source capable of exciting fluorophores positioned adjacent to the layer of conductive material and a light detector arranged to selectively detect emitted light that is generated by excited fluorophores, the detector being arranged to collect emitted light over a predetermined angular range relative to a surface of the first medium, said emitted light emanating from the layer of conductive material at the surface plasmon angle for an emission wavelength of the excited fluorophores relative to a surface of the layer of conductive material and passing through the first medium before being detected by the detector, the predetermined angular range comprising the surface plasmon angle for the emission wavelength of the excited fluorophores.

Another alternative exemplary method for observing surface plasmon-coupled emission can include arranging a first medium proximate to a light detector, the first medium having a layer of conductive material arranged on a surface thereof, the first medium having a first index of refraction and being a solid medium, said layer of conductive material being situated at an interface between said first medium and a second medium, the second medium having a second index of refraction different from the first index of refraction, the layer of conductive material comprising a patterned structure; causing one or more types of fluorophores to be adjacent to said layer of conductive material; exciting at least some of said fluorophores with an excitation source; and detecting emitted light that is generated by excited fluorophores with a detector, said emitted light having an emission wavelength of the fluorophores, said emitted light emanating from said layer of conductive material at the surface plasmon angle of said emission wavelength relative to a surface of said layer of conductive material and passing through said first medium before being detected by the detector.

In another alternative embodiment, an exemplary method of imaging fluorescence emission from one or more types of fluorophores bound to cellular sample can include: placing a cellular sample on a layer of conductive material disposed on a first medium, the first medium having a first index of refraction and being a solid medium, said layer of conductive material being situated at an interface between said first medium and a second medium, the second medium having a second index of refraction different from the first index of refraction; exposing said cellular sample to one or more substances capable of binding to one or more types of molecules in said cellular sample, said substances comprising one or more types of fluorophores, thereby causing fluorophores to be adjacent to said layer of conductive material; illuminating a selected position on said layer of conductive material at an excitation wavelength of said fluorophores; detecting emitted light that is generated by excited fluorophores at the selected position with a detector, said emitted light having an emission wavelength of the fluorophores, said emitted light emanating from said layer of conductive material at the surface plasmon angle of said emission wavelength relative to a surface of said layer of conductive material and passing through said first medium before being detected by the detector; and successively illuminating new selected positions on said layer of conductive material and detecting light emitted at each new selected position.

### BRIEF DESCRIPTION OF THE DRAWINGS

The objects and advantages of the invention will become apparent from the following detailed description of the preferred embodiments thereof in connection with the accompanying drawings, in which:

FIG. 1. Typical configuration for surface-plasmon resonance analysis. The incident beam is p-polarized.

FIG. 2. Propagation of light from a high refractive index medium (n_{P}) to a low refractive index medium n₀. For n₀=1, n_{P}=1.5, and θ_{P}=30.0, then θ₀=48.6°.

FIG. 3. Complex dielectrical constants for silver (top) and gold (bottom). Calculated from (Feldheim, D. L., and Foss, C. A. Jr. (Eds.), 2002, Overview. In Synthesis, Characterization, and Applications, Metal Nanoparticles, Marcel Dekker, Inc, New York. pp. 1-15; Johnson, B. P., and Christy, R. W., 1972, Optical constants of the noble metals, Rev. B. Condens, Matter 6: 4370; Born, M., and Wolf, E., 1980, Principles of Optics. Electromagnetic Theory of Propagation, Interference and Diffraction of Light, Pergamon Press, New York, 808 pp.; Yguerabide, J., and Yguerabide, E. E., 1998, Light-scattering submicroscopic particles as highly fluorescent analogs and their use as tracer labels in clinical and biological applications, Anal. Biochem., 262: 137-156).

FIG. 4. Schematic showing propagation constants in a prism and a thin film.

FIG. 5. Polarization definitions for light incident on a surface.

FIG. 6. Surface plasmon coupled emission. F is a fluorophore.

FIG. 7. Surface plasmon coupled emission with excitation by the evanescent wave (Kretschmann configuration) and from the side opposite the prism (reverse Kretschmann configuration).

FIG. 8. Surface plasmon coupled cone of emission for fluorophores near a metallic film.

FIG. 9. Cone of SPCE as seen from its central z axis. Top, wavelength distribution not drawn to scale. Bottom, p-polarization of SPCE.

FIG. 10. (a) Illustrates a Reverse Kretschman arrangement for SPCE that can result in suppression of background emission by observation of the plasmon-coupled emission. (b-d) Illustrate examples of patterned layers of conductive material. (e) Illustrates potential background suppression that can be achieved using SPCE.

FIG. 11. An example configuration for wavelength-ratiometric measurement using SPCE.

FIGS. 12A-12C. Exemplary arrangements for two-dimensional collection of emission using plasmon-coupled emission.

FIG. 13. An example arrangement for proximity-focused spectrofluorometer using a variable wavelength emission filter.

FIG. 14. An example arrangement of a prism spectrofluorometer using surface plasmon-coupled emission.

FIG. 15. An example arrangement for emission wavelength separation with grating-coupled emission.

FIG. 16. Exemplary potential geometries for efficient collection of SPCE.

FIG. 17. Fiber optics SPCE sensor.

FIG. 18. Structures of Cy3-DNA and Cy5-DNA.

FIG. 19. An example arrangement for directional fluorescence emission from hybridized DNA. Figure not drawn to scale, BSA-streptavidin ≈90°, ssCy3-DNA ≈70°.

FIG. 20. Fluorescence spectrum of dsCy3-DNa-biotin directional emission, SPCE, The insert shows an angular distribution of the fluorescence observed at 565 nm upon SP excitation at 514 nm.

FIG. 21. Angle dependent reflectivity of a silver film calculated according to published equations. This assumed values were dielectric constants of P=2.3, m=−13.5+0.5i for 565 nm, and m=−10.7+0.33i for 514 nm, dm=50 nm, 1=2.07, d1=15 nm, and 0=1.79.

FIG. 22. SPCE fluorescence observed at 565 nm (Cy3-DNA emission) upon injection of a ssCy3-DNA in presence (O) and absence (▴) of a complementary ssDNA-biotin deposited on the protein coated Ag 50 nm mirror.

FIG. 23. SPCE spectrum of dsCy3-DNA in presence of excess of ssCy5-DNA with surface plasmon (KR) excitation (--). Also shown is a free space emission observed in RK configuration. [dsCy3-DNA]=5.4×10-9 M and [ssCy5-DNA]=150×10-9 M.

FIG. 24. An example arrangement for binding of anti-Rabbit antibodies (labeled with Rhodamine Red-X) to Rabbit IgG immobilized on the silver surface. Non-binding anti-Mouse antibodies labeled with Alexa Fluor 647 remain in solution.

FIG. 25. An example arrangement for experimental geometry for measurements of free space and SPCE emission with reverse Kretschmann (RK) and Kretschmann (KR) configurations.

FIG. 26. Angular distribution of the 595 nm fluorescence emission of Rhodamine Red-X labeled anti-rabbit antibodies bound to the Rabbit IgG immobilized on the 50 nm silver mirror surface.

FIG. 27. Fluorescence spectra of the Rhodamine Red-X labeled anti-rabbit antibodies bound to the immobilized Rabbit IgG observed at 77° in RK-SPCE configuration; p and s refer to the orientation of the emission polarizer.

FIG. 28. Binding kinetics of the Rhodamine Red-X labeled anti-rabbit antibodies bound to Rabbit IgG immobilized on a 50 run silver mirror surface observed with KR/SPCE configuration (top). Bottom: emission spectra measured after 60 min.

FIG. 29. Emission spectra of the Rhodamine Red-X labeled anti-rabbit antibodies bound to Rabbit IgG immobilized on a 50 m silver mirror surface in presence of a fluorescent background (anti-Mouse antibodies labeled with Alexa Fluor 647) measured with different optical configurations.

FIG. 30. Fluorescence spectra (SPCE) of the Rhodamine Red-X labeled anti-rabbit antibodies bound to the Rabbit IgG immobilized on a 50 nm silver mirror surface in absence (---) and presence (- - -) of highly absorbing background (bovine Hemoglobin) observed with the KR/SPCE configuration.

FIG. 31. An example arrangement for two-color SPCE immunoassay.

FIG. 32. An example arrangement for experimental configuration for the two-color SPCE assay using surface plasmon (Kretschmann) excitation. Fibres F1 and F2 collect SPCE at 595 nm and 665 nm respectively. Fibre F3 observes the free-space emission.

FIG. 33. Calculated reflectivity of a 50 nm silver film on BK7 glass (np=1.52). The sample (protein layers) was assumed to be 15 nm thick (ns=1.50). The buffer thickness was taken as infinite with nw=1.33. For silver phase we used m532=−11.5+0.3i, m595=−15.0+0.4i and m665=−21.0+0.6i.

FIG. 34. Angle-dependent emission from a surface containing RhX-Ab and Alexa-Ab. Emission was measured at 595 or 665 nm. The sample was excited at 532 nm at 75° using the Kretschmann configuration.

FIG. 35. Angle-dependent emission from surface-bound RhX-Ab and Alexa-Ab measured at 595 and 665 nm. The sample was excited at 532 nm using the RK configuration.

FIG. 36. Emission spectra from a surface containing RhX-Ab and Alexa-Ab measured at three observations angles, using the KR configuration.

FIG. 37. Surface binding kinetics for the SPCE emission (▴,Δ) observed as shown in FIG. 31 and FIG. 34, at 71° for 595 nm and −68° for 665 nm. Free-space emission (●,∘).

### DETAILED DESCRIPTION OF EXEMPLARY EMBODIMENTS OF THE INVENTION

A new approach is described for collection of fluorescence which can provide 50% light collection efficiency using simple and inexpensive optics. This new opportunity in fluorescence detection is based on the interaction of excited state fluorophores with a nearby continuous metallic surface. Fluorophores above a metal surface can couple with the plasmon resonances in the surface, resulting in directional and wavelength-resolved emission. Surface plasmon-coupled emission (SPCE) is a reverse process of surface plasmon resonance (SPR) as seen in the angle-dependent absorption of thin metal films.

This novel utilization of fluorophore-metal interactions promises to have numerous applications in the biosciences. Below, how this interaction can result in efficient light collection and directional emission is shown. Then, potential applications of this useful phenomenon are described.

Surface Plasmon Resonance

Surface Plasmon Resonance Analysis

The phenomenon of surface plasmon resonance provides an approach for understanding SPCE. The term surface plasmon resonance can refer to the phenomenon itself (SPR) or to the use of this phenomenon to measure biomolecule binding to surfaces. To avoid confusion we refer to this latter use of SPR as surface plasmon resonance analysis, SPRA, This method is now widely used in the biosciences and provides a generic approach to measurement of biomolecule interactions on surfaces (Salamon, Z., Macleod, H. A., and Tollin, G., 1997, Surface plasmon resonance spectroscopy as a tool for investigating the biochemical and biophysical properties of membrane protein systems. I: Theoretical principles, Biochim. et Biophy. Acta. 1331: 117-12; Melendez, J., Carr, R., Bartholomew, D. U., Kukanskis, K., Elkind J., Yee, S., Furlong, C., and Woodbury, R., 1996, A commercial solution for surface plasmon sensing, Sensors and Actuators B, 35-36: 212-216; Liedberg, B., and Lundstrom, I., 1993, Principles of biosensing with an extended coupling matrix and surface plasmon resonance, Sensors and Actuators B, 11: 63-72; Cooper, M. A., 2002, Optical biosensors in drug discovery, Nature Reviews, 1: 515-528; Wegner, G. J., Lee, H. J., and Corn, R. M., 2002, Characterization and optimization of peptide arrays for the study of epitope-antibody interactions using surface plasmon resonance imaging, Anal. Chem., 74: 5161-5168).

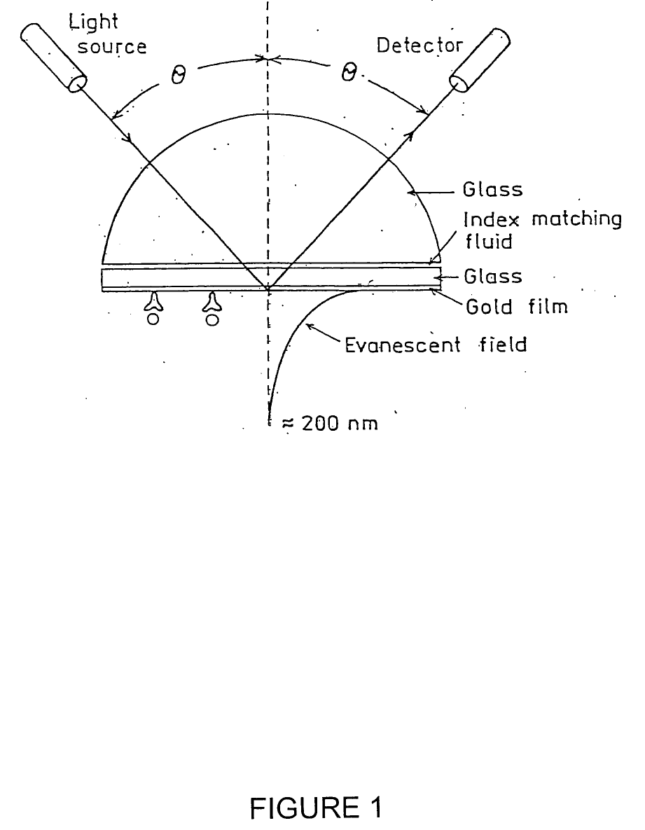

A schematic description of SPRA is shown in FIG. 1. The measurement is based on the interaction of light with thin metal films on a glass substrate. The film is typically made of gold 40-50 nm thick. The analysis surface consists of a capture biomolecule which has affinity for the analyte of interest. The capture biomolecule is typically covalently bound to the gold surface. The analysis substrate is optically coupled to a hemispherical or hemicylindrical prism by an index matching fluid. Light impinges on the gold film through the prism, which is called the Kretschmann configuration. The instrument measures the reflectivity of the gold film at various angles of incidence (θ), with the same angle used for observation (θ). Other configurations can be used, such as a triangular prism or more complex optical geometry and a position-sensitive detector. In any event the measurement is the same, reflectivity of the gold surface versus angle of incidence.

The usefulness of SPRA is due to the large dependence of the gold film reflectivity on the refractive index of the solution immediately above the gold film. Binding of macromolecules above the gold film causes small changes in the refractive index which result in changes in reflectivity. Typical SPRA data in the form of a plot of reflectivity versus the angle of incidence for a gold film can be found in the literature. (Frutos, A. G., and Corn, R. M., 1998, SPR of utrathin organic films, Analytical Chemistry, 449A-455A; Jordan, C. E., Frey, B. L., Kornguth, S., and Corn, R. M., 1994, Characterization of Poly-L-lysine adsorption onto alkanethiol-modified gold surfaces with polarization-modulation fourier transform infrared spectroscopy and surface plasmon resonance measurements, Langmuir, 10: 3642-3648; Frey, B. L., Jordan, C. E., Kornguth, S., and Corn, R. M., 1995, Control of the specific adsorption of proteins onto gold surfaces with poly(1-ysine) monolayers, Anal. Chem., 67: 4452-4457). According to the published reports, a reflectivity minimum occurs at the SPR angle. The SPR angle was reported to change as the gold surface is coated with 11-mercaptoundecanoic acid (MU), then biotinylated poly-lysine (PL) and finally avidin. The changes in SPR angle has been attributed to changes in refractive index near the gold surface due to the adsorbed layers.

The decrease in reflectivity at the SPR angle (θ_{SP}) is due to absorption of the incident light at this particular angle of incidence. At this angle the incident light is absorbed and excites electron oscillations on the metal surface. The reflectivity is sensitive to the refractive index of the aqueous medium although the light is reflected by the gold film. This sensitivity is due to an evanescent field which penetrates approximately 200 nm into the solution (FIG. 1). The evanescent field appears whenever there is resonance between the incident beam and the gold surface, and is not present when there is no plasmon resonance, that is, where the reflectivity is high.

The existence of an evanescent field is reminiscent of total internal reflectance (TIR) which occurs between a glass-water interface when the angle of incidence from the glass slide exceeds the critical angle (Axelrod, D., Hellen, E. H., and Fulbright, R. M., 1992, Total internal reflection fluorescence, in Topics in Fluorescence Spectroscopy, Vol 3: Biochemical Applications, (Lakowicz, J. R., Ed), Plenum Press, New York, 3: 289-343). There is often confusion about the relationship between the critical angle in TIR (θ_{C}) and the SPR angle (θ_{SP}). The physical origins of θ_{C} and θ_{SP} are similar, but these angles are different and not directly related. Comparing glass and silver-coated glass surfaces, the silver-coated surface shows high reflectivity at all angles except near the plasmon angle of about 30°. The reflectivity of a glass surface is quite different. The reflectivity is low below the critical angle θ_{C}, increases sharply to nearly 100% at θ_{C}, and the reflectivity remains high for all angles above θ_{C}. For the glass surface and angles above θ_{C} there exists an evanescent field from the totally internally reflected light. For the silver-coated glass there is no evanescent field in the aqueous phase unless the angle of incidence is near the SPR angle. The reflectivity of the silver film is high at angles significantly larger or smaller than θ_{SP}.

The evanescent wave due to SPR is much more intense than that due to TIR. The relative strengths of the fields can be measured by the fluorescence from fluorophores near the surface. Fluorophores can be localized within the evanescent field by coating with a polyvinyl alcohol (PVA) film containing a fluorophore (Neumann, T., Johansson, M. L., Kambhampati, D., and Knoll, W., 2002, Surface-plasmon fluorescence spectroscopy, Adv. Funct. Mater, 12: 9-575-586). The dependence of the emission on incident angle indicates the relative intensity of the evanescent wave felt by the fluorophores. For the glass surface the emission intensity is low for θ<θ_{C}. This low value is essentially the same as seen in a typical fluorescence measurement where the fluorophore is excited in a glass or quartz cuvette. As the incident angle exceeds θ_{C} the intensity drops about 2-fold because the incident light undergoes TIR rather than passing into the sample. Above the critical angle the remaining intensity represents the amount of excitation due to the TIR evanescent wave. This result indicates the field strength for TIR is roughly the same for the incident light and the evanescent wave. For clarity we note that the intensity on glass seen for θ<θ_{C} will increase with the thickness of the PVA film. The intensity for θ>θ_{C} will be mostly independent of the film thickness once it exceeds the penetration depth of the evanescent wave.

Remarkably, different results are seen for the labeled film on the silver surface. The emission intensity is near zero for angles above and below θ_{C} because of the high reflectivity of the metal film. In contrast to uncoated glass, the light does not penetrate the sample even though 0<θ_{C}. There is a dramatic increase in the emission intensity of the film near the plasmon angle of about 15-fold. This effect is due to a 10 to 40-fold increase in the intensity of the evanescent field above silver as compared to above glass with TIR (Liebermann, T., and Knoll, W., 2000, Surface-plasmon field-enhanced fluorescence spectroscopy, Colloids and Surfaces, 171: 115-130; Attridge, J. W., Daniels, P. B., Deacon, J. K., Robinson, G. A., and Davidson, G. P., 1991, Sensitivity enhancement of optical immunosensors by the use of a surface plasmon resonance fluoroimmunoassay, Biosensors and Bioelectronics 6: 201-214; Liebermann, T., Knoll, W., Sluka, P., and Herrmann, R., 2000, Complement hybridization from solution to surface-attached probe-oligonucleotides observed by surface-plasmon-field-enhanced fluorescence spectroscopy, Colloids and Surfaces 169: 337-35; Fukuda, N., Mitsuishi, M., Aoki, A., and Miyashita, T., 2002, Photocurrent enhancement for polymer Langmuir-blodgett monolayers containing ruthenium complex by surface plasmon resonance, J. Phys. Chem. B, 106: 7048-7052; Attridge, J. W., Daniels, P. B., Deacon, J. K., Robinson, G. A., and Davidson, G. P., 1991, Sensitivity enhancement of optical immunosensors by the use of a surface plasmon resonance fluoroimmunoassay, Biosensors and Bioelectronics, 6: 201-214). This increase in field strength above a metal film is one origin of the increased sensitivity possible with plasmon-coupled emission.

A characteristic of the SPR angles is that they are strongly dependent on wavelength. For example, reflectivity curves of a gold film at several wavelengths (Natan, M. J., and Lyon, L. A., 2002, Surface plasmon resonance biosensing with colloidal Au amplification, in Metal Nanoparticles: Synthesis, Characterization, and Applications (Feldheim, D. L., and Foss, C. A, Ed.), Marcel Dekker, New York, pp 183-205) show that the surface plasmon angle decreases as the wavelength decreases. The dependence on wavelength can be understood in terms of the optical constants of the metals, which depend upon wavelength (frequency) and the dielectric constant of the adjacent prism. This dependence of θ_{SP} on wavelength is the origin of intrinsic spectral resolution when observing surface plasmon-coupled emission.

The theory of SPR can provide a basis for understanding SPCE. Prior to describing the theory of SPR it is informative to understand the angular shift in θ_{SP} measuring during SPRA. A scan of the SPRA literature shows that in most experiments the changes upon biomolecule binding are reported in relative units (RU) (Malmqvist, M., 1999, BIACORE: an affinity biosensor system for characterization of biomolecular interactions, Biochemical Society Transactions 27: 335-340; Gestwicki, J. E., Hsieh, H. V., and Pitner, J. B., 2001, Using receptor conformational change to detect low molecular weight analytes by surface plasmon resonance, Anal. Chem., 73: 5732-5737; Hendrix, M., Priestley, E. S., Joyce, G. F., and Wong, C. H., 1997, Direct observation of aminoglycoside-RNA interactions by surface plasmon resonance, Journal of the American Chemical Society, 119: 16-3636-3648; Woodbury, R. G., Wendin, C., Clendenning, J., Melendez, J., Elkind, J., Bartholomew, D., Brown, S., and Furlong, C. E., 1997, Construction of biosensors using a gold-binding polypeptide and a miniature integrated surface plasmon resonance sensor, Biosensors and Bioelectronics 13: 1117-1126; Kolomenskii, A. A., Gershon, P. D., and Schuessler, H. A., 1997, Sensitivity and detection limit of concentration and adsorption measurements by laser-induced surface-plasmon resonance, Applied Optics, 36: 25-6539-6547). The change in RU is typically measured during a binding reaction, in an exemplary case, binding of bovine-serum albumin (BSA) to a dextran-coated gold surface. The sample is initially washed with buffer. Washing with bovine serum albumin (BSA) causes a change of about 1 kRU, which can be reversed by washing with buffer. This change is due to the effect of BSA on the refractive index of the solvent. To determine the effect of surface-bound BSA the dextran was activated with coupling reagents (NHS/EDC), incubated with BSA, then washed with buffer. Covalent coupling of BSA to the surface results in a non-reversible change of about 1 kRU. Even this moderately dense coating of protein results in a change of just 1 kRU. 1 RU is defined as a shift in the SPR angle of 10⁻⁴ degrees (Kolomenskii et al. 1997, supra). Hence, the changes in θ_{SP} during SPRA are very small, typically 0.1 degrees. As will be shown below, this small change in SPR with biomolecule binding indicates that surface binding reactions will not interfere with the intrinsic spectral resolution of plasmon-coupled emission.

Theory of Surface Plasmon Resonance

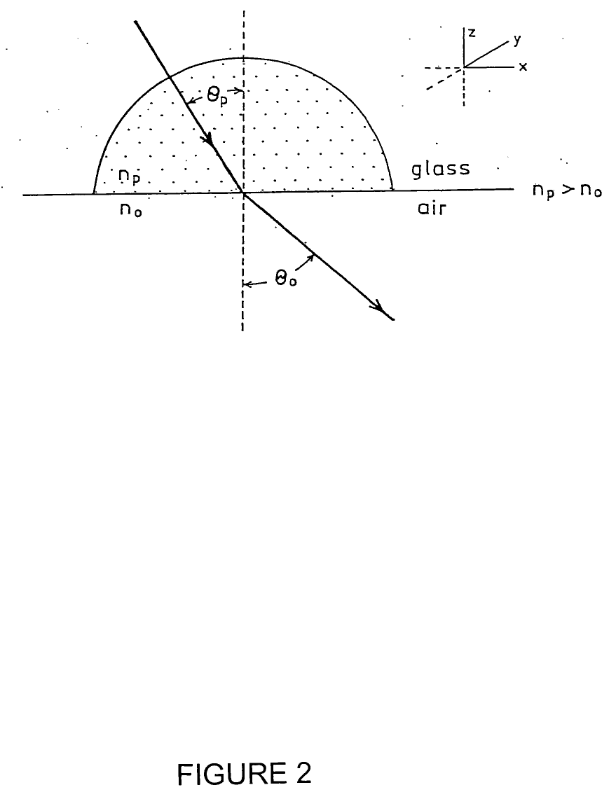

Knowledge of the theory of SPR is useful for understanding surface plasmon-coupled emission. To understand SPR it is helpful to review the physical origin Snell's law. FIG. 2 shows the propagation of light across an interface where the dielectric constant of the prism (n_{P}) is greater than the air (n₀). The angles of the incident (θ_{P}) and refracted (θ₀) beams are related by Snell's law.
nₚ sin θₚ=n₀ sin θ₀ (1)

For example, suppose θ_{P}=30°, and that the index of refraction is typical of glass, n_{P}=1.5, then θ₀=48.6°. As the angle of incidence (θ_{P}) increases the component of the beam along the x axis in the prism also increases. It is easy to visualize the rapid increase in the x component of the field as θ_{P} increases.

TIR occurs when the refracted beam can no longer propagate in air. The largest possible component of the beam in the air along the x axis is given when θ₀=90°, which occurs when θ_{P} is a smaller angle less than 90°. When θ₀=90° the sin θ₀=1.0 and the incident angle from medium 2 is given by

$$sin {θ}_{P} = \frac{{n}_{0}}{{n}_{P}} = sin {θ}_{C} \qquad ( 2 )$$

where θ_{c} is called the critical angle for TIR. For an angle of incidence equal to θ_{C} the x-component is infinite. For θ>θ_{C} no angle satisfies Eq. 2 (sin θ₀>1) and the incident beam is reflected back into the denser medium. For the case in question, the critical angle is 41.8°. If one attempts to use θ_{P} greater than 41.8° in Eq. 1 there is no solution for θ₀ since the calculated value of sin θ₀>1.0.

The phenomenon of refraction and TIR can be understood in terms of the Maxwell's equation (Sambles, J. R., Bradbery G. W., and Yang, F., 1991, Optical excitation of surface plasmons: an introduction, Contemporary Physics, 32: 3-173-183; Levi, L., 1968, Applied Optics. A Guide to Optical System Design/Volume 1, John Wiley & Sons, New York, 620 pp; Born, M., and Wolf, E., 1980, Principles of Optics. Electromagnetic theory of propagation, interference and diffraction of light, Pergamon Press, New York, pp. 808). An electromagnetic wave propagating in space can be described by
\overline{E}(\overline{r},t)=\overline{E₀} exp(iωt−i\overline{k}·\overline{r}) (3)
where the bars indicate vector quantities, \overline{r} is a unit vector in the direction of propagation, ω is the frequency in radians/sec. The term \overline{k} is the propagation constant which is sometimes called the wavevector. This value is given by

$$k = \frac{2 π}{λ} = \frac{n ω}{c} = {nk}_{0} \qquad ( 4 )$$

where λ is the wavelength, n is the refractive index of the medium and k₀ is the propagation constant of the wave in a vacuum. It is understood that the physical values are given by the real part of Eq. 3. Hence the electric field is described by
\overline{E}(r,t)=\overline{E₀} exp(cos ωt−\overline{k}−·\overline{r}) (5)

For TIR, consider the electric field along the x-axis, the prism-water interface. This component is given by
E(x,t)=E₀ₓ exp(cos ωt−kₓx) (6)
In order to satisfy Maxwell's equations the electric fields have to be continuous across the interface, which requires kₓ to be equal in both media. Hence
kₚ sin θₚ=k₀ sin θ₀ (7)
Since k_{P}=n_{P} ω/c and k₀=n₀ ω/c continuity across the interface requires the angles be related according to Eq. 1.

Surface plasmon resonance can also be understood by continuity of the electric field across the interface. However, we need to consider the complex optical properties of metallic surfaces. It is well known that if an electrical field E₀ is incident on a dielectric material the field within the material is E=εE₀ where ε is the dielectric constant. For a dielectric the refractive index n is often related to the dielectric constant by n=\sqrt{ε}. This relationship holds if n and ε are measured at the same frequency. For polar liquids one often finds because the dielectric constant is measured at lower frequencies where the polar molecules can reorient in the electric field.

The optical constants n and s are more complex for metals, in fact they are described by imaginary numbers. In a dielectric all the electrons are bound to the nuclei. In a metal some of the electrons are free and can respond to an incident field. At low frequencies the metal is a conductor. At higher frequencies the electrons oscillate in response to the oscillating incident field. While the electrons in a metal are highly mobile, they are not infinitely fast. The rate of electron motion in response to an applied field can be understood in terms familiar to time-resolved spectroscopy. Suppose the electrons are moving with a velocity v₀ in response to an electric field. When the field is turned off the velocity decays exponentially as v=v₀ exp (−βt) where is the decay constant with a value near 3×10¹³ sec⁻¹ (Born, M., and Wolf, E., 1980, Principles of Optics. Electromagnetic theory of propagation, interference and diffraction of light, Pergamon Press, New York, pp. 808; Crawford, F. S., Jr., 1968, Waves, McGraw-Hill Publishers, New York, 600 pp). The relaxation times (1/β) are very fast and are near 0.03 ps=30 fs. Consider light with a wavelength of 500 nm, which corresponds to a frequency of 0.6×10⁻¹⁵ sec⁻¹. The electrons respond to the electric field but cannot keep up completely, which would happen at longer wavelengths where the frequency is lower. At shorter wavelengths or higher frequencies the electrons cannot respond and the material may become transparent if other absorption bands are not present. The optical and reflective properties of silver and gold depend on the interplay of incident frequency and electron mobility, as well as underlying absorption bands not relate to electron oscillations.

This interplay of electron mobility and incident frequency results in complex and imaginary optical constants. The refractive index and dielectric constants of a metal are given by
nₘ=nᵣ+inᵢₘ (8)
εₘ=εᵣ+iεᵢₘ (9)
where subscripts indicate the real (r) and imaginary (im) components. These constants are wavelength (frequency) dependent. Some intuition about the physical meaning of these terms can be obtain from examining specific examples (Feldheim, D. L., and Foss, C. A. Jr. (Eds.), 2002, Overview. In Synthesis, Characterization, and Applications, Metal Nanoparticles, Marcel Dekker, Inc, New York. pp. 1-15; Johnson, B. P., and Christy, R. W., 1972, Optical constants of the noble metals, Rev. B. Condens, Matter 6: 4370; Born, M., and Wolf, E., 1980, Principles of Optics. Electromagnetic Theory of Propagation, Interference and Diffraction of Light, Pergamon Press, New York, 808 pp.; Yguerabide, J., and Yguerabide, E. E., 1998, Light-scattering submicroscopic particles as highly fluorescent analogs and their use as tracer labels in clinical and biological applications, Anal. Biochem., 262: 137-156) of gold and silver, which are expected to be most useful metals for SPCE. The dielectric constants for gold and silver can be calculated according to published equations. (Feldheim, D. L., and Foss, C. A. Jr. (Eds.), 2002, Overview. In Synthesis, Characterization, and Applications, Metal Nanoparticles, Marcel Dekker, Inc, New York. pp. 1-15). The imaginary part of the dielectric constant is small and positive. The imaginary part is related to light absorption, which can be seen by the larger values of εᵢₘ of gold for wavelengths below 500 nm. The real part of εₘ becomes increasingly more negative as the wavelength increase. This effect can be interpreted as electron oscillations with the charge opposite to the incident field. As the incident frequency decreases εᵣ becomes more negative reflecting more complete response of the electrons to the lower frequency. For a perfect conductor εᵣ approaches minus infinity.

The phenomenon of SPR can be understood by considering the propagation constant of the electromagnetic wave in the metal along the x-axis. In the metal film the field is described by Eqs. 5 and 6 with kₓ=kᵣ+i kᵢₘ being the complex wavevector along the x axis. For a metal the propagation constant for the surface plasmon is given by

$${k}_{sp} = {\frac{ω}{c} [ \frac{{ɛ}_{m} {ɛ}_{p}}{{ɛ}_{m} + {ɛ}_{p}} ]}^{1 / 2} = {{k}_{0} [ \frac{{ɛ}_{m} {ɛ}_{p}}{{ɛ}_{m} + {ɛ}_{p}} ]}^{1 / 2} \qquad ( 10 )$$

where εₘ and εₚ are the dielectric constant of the metal (m) and prism (p), respectively. Because the real part of εₘ is larger than the imaginary part the propagation constant can be approximated by

$${k}_{sp} = {{k}_{0} [ \frac{{ɛ}_{r} {ɛ}_{p}}{{ɛ}_{r} + {ɛ}_{p}} ]}^{1 / 2} \qquad ( 11 )$$

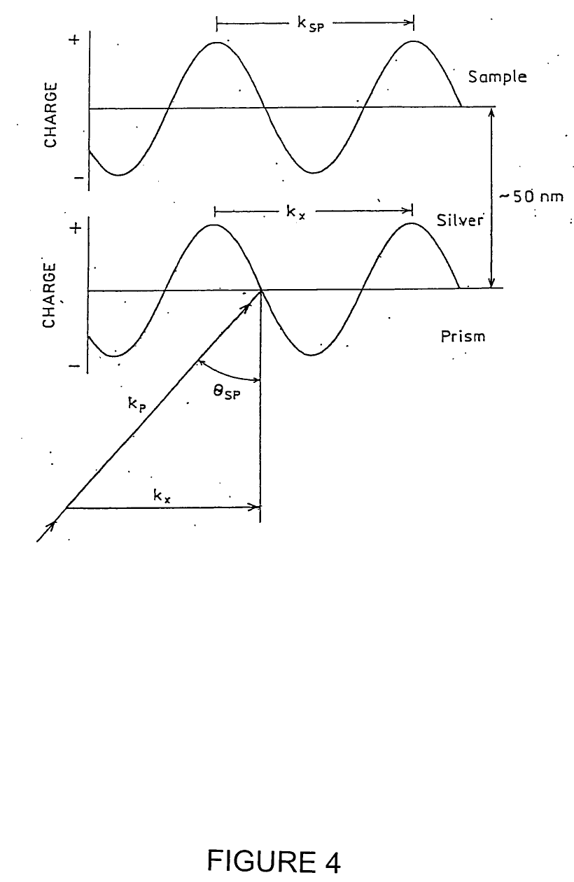

The incident light can excite a surface plasmon when its x-axis component equals the propagation constant for the surface plasmon (FIG. 4). The propagation constant for the incident light in the prism (p) is given by
kₚ=k₀nₚ (12)
and the component along the x-axis is equal by
kₓ=k₀nₚ sin θₚ (13)
where θₚ is the incidence angle in the prism. Hence the conditions for SPR absorption is satisfied when
kₛₚ=kₓ=k₀nₚ sin θₚ (13)
These considerations show that the surface plasmon resonance occurs whenever the x-axis component of the incident field equals that obtained from Eq. 11.

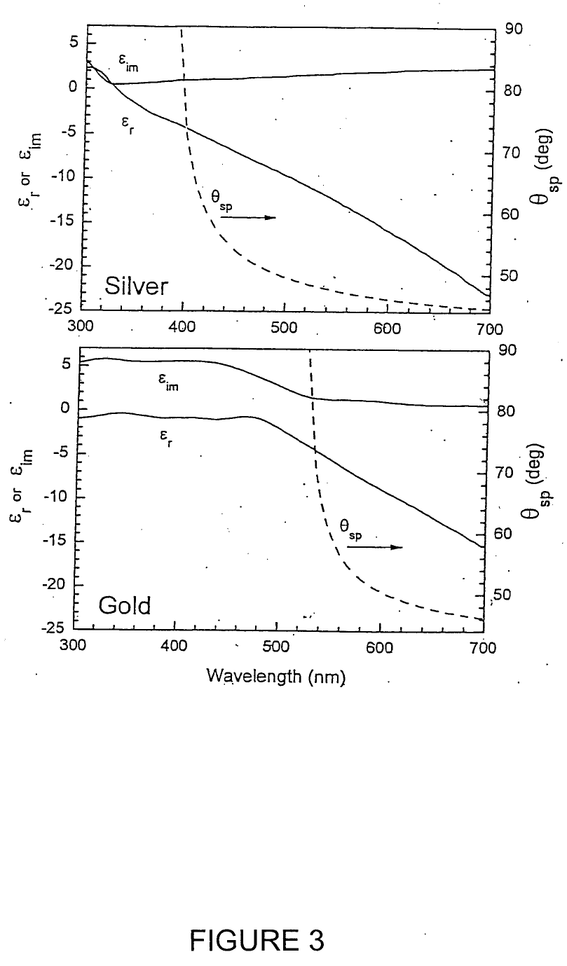

Detailed consideration of Eq. 10 yields some interesting insights. Suppose light is incident on the metal from a vacuum or air (n=1.0). The maximum value of kₓ is given when θ_{P}=0, yielding kₓ=k₀. Examination of FIG. 3 shows that the real parts of εₘ are negative and much greater than one, these parts dominate the ratio in Eq. 10 so that k_{SP} is always larger than the free space wave vector k₀. For this reason surface plasmons cannot be excited with light incident from the air or medium with the lower dielectric constant. The large value of kₛₚ>k is due to the finite speed of electron motion which makes the metal less than a perfect conductor at optical frequencies.

In order to obtain SPR the magnitude of kₓ must be increased to equal or exceed k_{SP}. This can be accomplished using the configuration shown in FIG. 1 where light is incident on the metal film from the prism side. This approach increases the wave vector to kₚ=nₚk₀. This results in the maxima and minima of the electric field being more closely spaced, but now k_{SP} is less than k₀. To obtain resonance the x-components of the electric field distribution are then matched by adjustment of the x-component of k_{P} by a factor sin θ_{P} (FIG. 4).

The value of θ_{SP} can be calculated using Eqs. 11-14. These values are shown on FIG. 3. The values of θ_{SP} increase with decreasing wavelengths. Because of the wavelength-dependent dielectric constants there is a lower wavelength limit below which θ_{SP} cannot be calculated. Above this lower wavelength limit the dependence of θ_{SP} on wavelength is roughly the same for silver and gold.

An intuitive approach to explaining the resonances at different angles and wavelengths can be appreciated by considering a mental picture of the interaction by considering the wavelength of the incident light in the prism, and the projection of this distance onto the interface (FIG. 4). SPR occurs when this projected distance matches the wavelength of the surface plasmon. This visualization of SPR explains the increase in θ_{P} needed for resonance at shorter wavelengths. However the increase in θ_{P} needed for an increase in wavelength is offset in part by the wavelength-dependent properties of the metal.

While one can readily calculate θ_{SP} it is considerably more difficult to calculate reflectivity curves. This requires calculation of the reflectivity of the film for a range of incidence angles. Additionally it is necessary to recall that a SPRA experiment involves at least three phases, the prism, metal and solution containing the analyte. If one considers the region of the sample beyond the surface-bound macromolecules then one has to consider four phases. The equations are complex and have been published (Pockrand, I., 1978, Surface plasma oscillations at silver surfaces with thin transparent and absorbing coatings, Surface Science, 72: 577-588; Kurihara, K., and Suzuki, K., 2002, Theoretical understanding of an absorption-based surface plasmon resonance sensor based on Kretschmannn's theory, Anal. Chem., 74: 696-701; Salamon, Z., Macleod, H. A., and Tollin, G., 1997, Surface plasmon resonance spectroscopy as a tool for investigating the biochemical and biophysical properties of membrane protein systems. I: Theoretical principles, Biochimica et Biophysica Acta, 1331: 117-129; Homola, J., Koudela, I., and Yee, S. S., 1999, Surface plasmon resonance sensors based on diffraction gratings and prism couplers: sensitivity comparison, Sensors and Actuators B, 54: 16-24; Brockman, J. M., Nelson, B. P., and Corn, R. M., 2000, Surface plasmon resonance imaging measurements of ultrathin organic films, Annu. Rev. Phys. Chem, 51: 41-63; Nelson, B. P., Frutos, A. G., Brockman, J. M., and Corn, R. M., 1999, Near-infrared surface plasmon resonance measurements of ultrathin films. 1. Angle shift and SPR imaging experiments, Anal. Chem. 71: 3928-3934). A convenient resource is at corninfo.chem.wisc.edu. This web site calculates reflectivity curves for any chosen optical constants and metal thickness for a 3 or 4 phase system. Reflectivity curves can also be calculated using commercial software as known to those of ordinary skill in the art.

Polarization Relative to a Surface

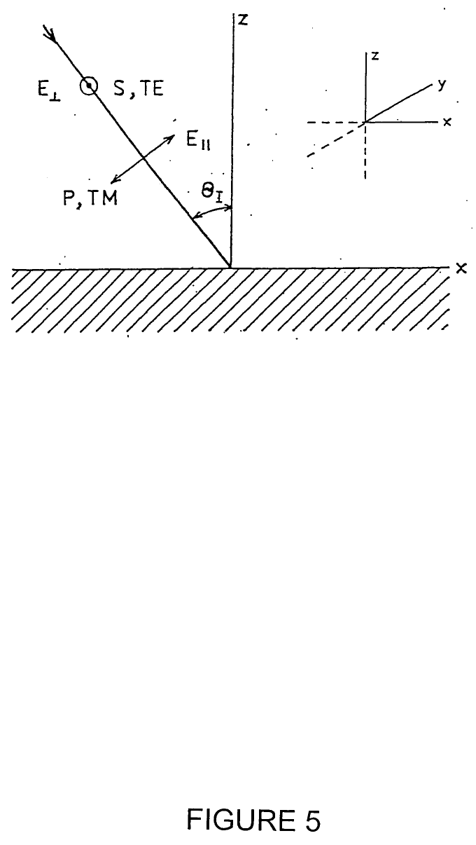

For individuals familiar with fluorescence polarization or anisotropy measurements the conventions used with surfaces can be confusing. For fluorescence measurements in a cuvette the symmetry axis is the z-axis so measurements of the polarized intensities are made relative to this axis. For light incident on a surface the symmetry axis is the plane of incidence formed between the incident ray and an axis normal to the surface, which is in the plane of the paper in FIG. 5. An incident ray is said to be p-polarized if the electric vector (E_{∥}) is parallel to the plane of incidence. This polarization is also referred to as TM polarized, meaning the magnetic vector is transverse to the plane of incidence. An incident beam is said to be s-polarized (E⊥) when the electric vector is perpendicular to the plane of incidence. Such a beam is also described as TE-polarized, meaning the electric vector is transverse to the plane of incidence. For p-polarized light it is easy to see that the interaction of the electric field with the metal surface depends on θ_{I}.

When measuring or discussing SPR one may assume the incident beam is p-polarized. It is the p-polarized component of the beam that gives the reflectivity curves. The s-polarized beam will not excite the surface plasmon and will not show decreased reflectivity at some angle of incidence. The origin of this difference can be understood by considering the interactions of the effective field with the metal surface. For the s-polarized component the electric field does not depend on θ_{I}.

Fluorescence and Thin Metal Films

Surface Plasmon-Coupled Emission

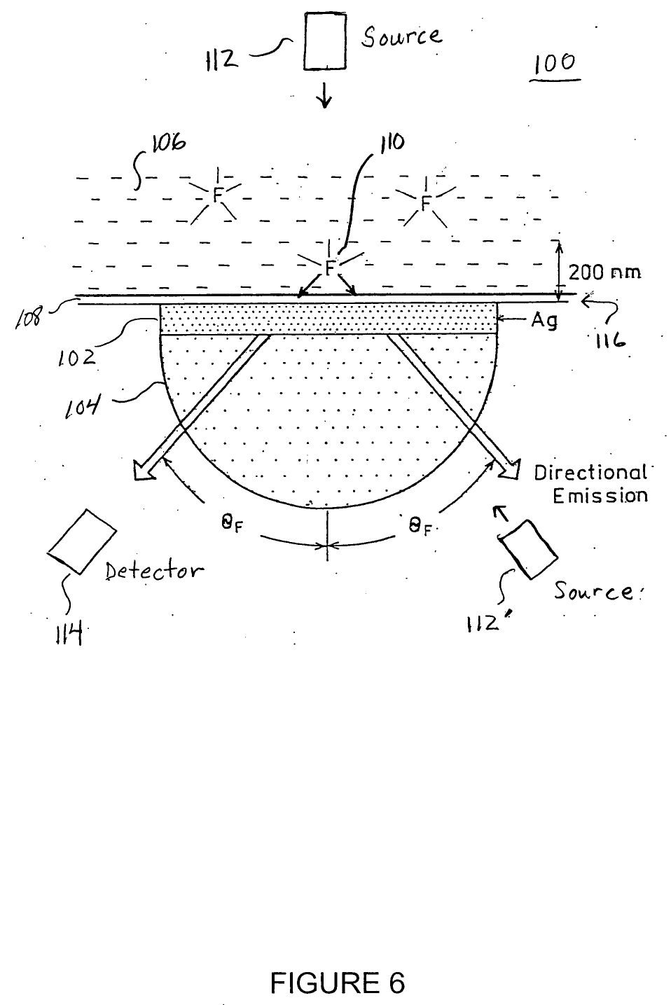

Now consider how the phenomenon of SPR can be applied to fluorescence detection. The plasmon angle is different for different wavelengths. The reflection minima are more strongly dependent on wavelength when compared to the effects of biomolecule binding. If an incident beam can excite a surface plasmon and create an evanescent field it seems logical that an excited fluorophore can excite a surface plasmon and create a radiative beam (FIG. 6). This phenomenon will be called surface plasmon-coupled emission, SPCE, Proximity of a fluorophore to a metallic film can result in the emission becoming directional with sharply defined angles (θ_{F}). These angles are different from θ_{SP} when the excitation and emission wavelengths are different. For example, suppose a fluorophore was excited at 633 nm and observed at 750 nm. From FIG. 4 it can be seen that if the fluorescence is excited at the reflectivity minimum for 633 nm at θ_{SP}=43.7° the emission would couple back into the prism with θ_{F}=42.7° which is the reflectivity minimum for 750 nm. This difference of 1° may not sound large but recall that the average change in θ_{SP} in a SPRA measurement is 0.1°.

The dependence of θ_{F} on wavelength suggests that different fluorophores will display directional emission at different angles determined by the emission maxima. If SPCE displays the same characteristics as SPR, then fluorophores near the metal film will emit into the prism at angles defined by the optical properties of the metal, i.e. the SPR angle for the emission wavelength. This coupling should occur for fluorophores within the region where the evanescent waves exist, out to a SPCE coupling range of about 200-500 nm from the metal surface.

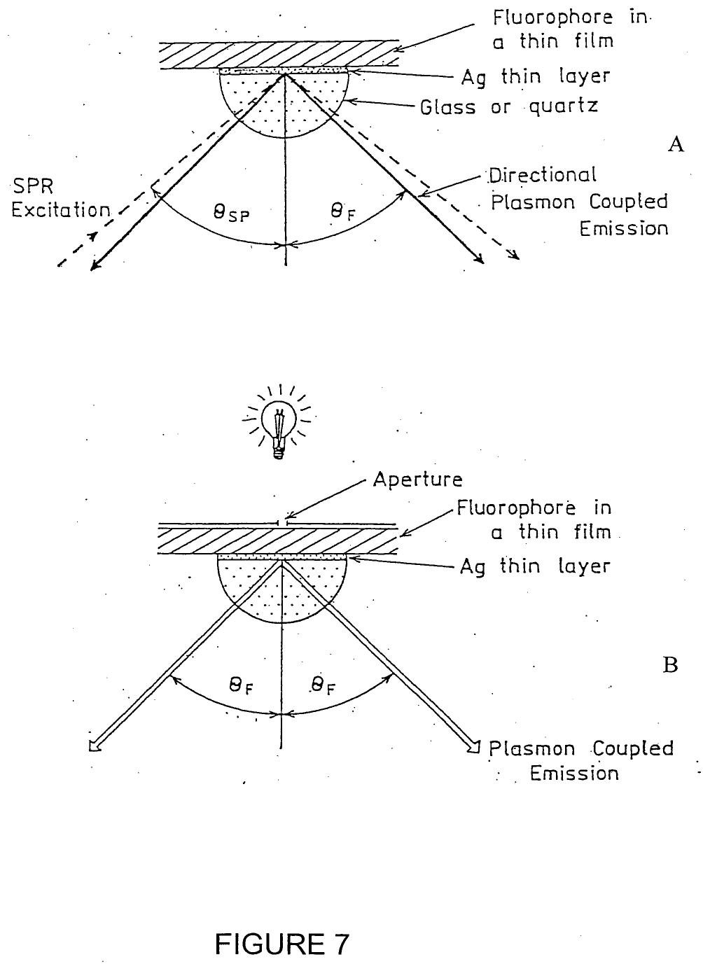

Using the analogy with SPR we can make several other predictions about SPCE. In the case of SPRA the metal film is illuminated with an incident beam which defines symmetry across the plane of incidence. Illumination of the metal through the prism is called the Kretschmann configuration. The fluorophores will be excited by the evanescent field in the sample which exists when θ_{I}=θ_{SP} Hence only fluorophores with a fractional wavelength distance from the metal will be excited. Based on the wavelength dependence of reflectivity we expect the longer wavelength Stokes' shifted emission to occur at smaller angles from the z-axis (FIG. 7, top). Based on recent experiments (Gryczynski, I., Malicka, J., and Lakowicz, J. R., 2003, Radiative Decay Engineering 4. Experimental studies of surface plasmon-coupled emission, Anal. Biochem,) and a reading of the literature it is expected that θ_{F} will be independent of the distance from the metal surface, except to the extent due to the thickness and dielectric constant above the metal film.

Suppose the sample is excited from the side opposite the prism which is called the “reverse Kretschmann” configuration. This mode of excitation is shown in FIG. 7 (bottom) where a small spot is illuminated by a light source and a small aperture in a spatial filter. In this case the incident light would not create surface plasmons and there is no evanescent field due to the incident light. An excited fluorophore near the metal film would not know how it was excited, that is, the emission should be the same whether the fluorophore is excited by evanescent SPR field (top) or from a light source not coupled to the surface plasmon (bottom). In the reverse Kretschmann configuration the fluorophores will be excited uniformly across the thickness of the sample. However, only those fluorophores within a fractional wavelength distance from the metal will couple to the surface and result in SPCE. This allows the use of SPCE without the surface plasmon excitation, which can simplify the devices based on this phenomenon. Since fluorophores at distances greater that the plasmon coupling range from the metal will not couple, autofluorescence from molecules not localized by the surface chemistry can be suppressed by selectively detecting coupled emission.

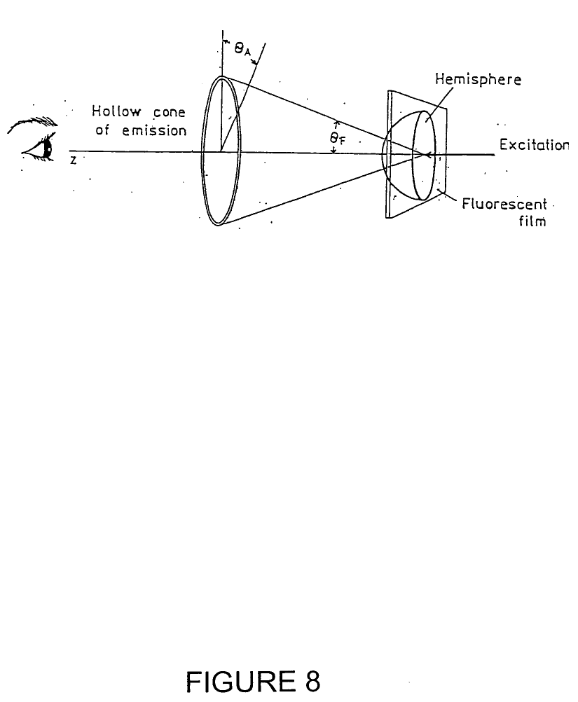

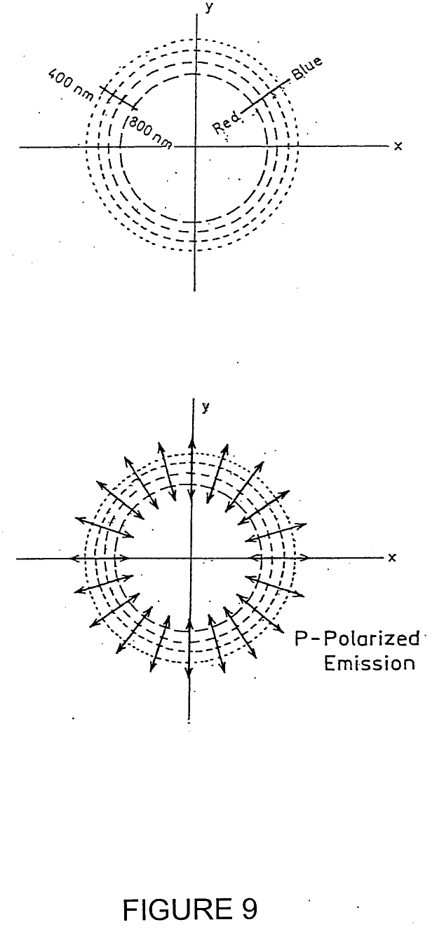

Consider excitation from the sample phase (FIG. 7, bottom). Since the fluorophore emits without remembering its source of excitation, there is no plane of incidence for the plasmon-coupled emission. The SPCE will be the same for all azimuthal angles (θ_{A}) around the z-axis. Suppose the prism is a hemisphere rather than a hemicylinder (FIG. 8). The emission seen looking along the z-axis will appear as a cone at angle θ_{F} with an equal distribution of emission at all angles θ_{A}. Additionally, different emission wavelengths will appear at different cone angles, that is, a multi-color sample will display a rainbow-like pattern (FIG. 9, top) with the longer wavelength being inside the longer wavelengths.

Plasmon-coupled emission provides useful polarization properties. Recall that SPR occurs for the p-polarized component of the incident light because the interaction of the electric field with the metal plane depends on the incident angle. Similarly, the p-polarized component of the emission is expected to couple with the surface plasmons. Hence, the polarization of the cone will always point away from the normal z-axis (FIG. 9, bottom), that is the SPCE will be p-polarized at all angles around the cone, independently of the mode of excitation. The SPCE will remain p-polarized whether the sample is fluid or solid, that is, whether or not the free space emission of the sample is lower or high. The coupling efficiency with the surface plasmons will depend on fluorophore orientations relative to the metal surface. Some dipoles with the s-orientation may couple into the plasmon, but much more weakly. It is not clear if this smaller amount of coupled emission will be s or p polarized in the prism.

Theoretical and Experimental Studies of Plasmon-coupled Emission

There are a considerable number of publications on the theoretical aspects of fluorophores interacting with metallic surfaces or mirrors (Ford, G. W., and Weber, W. B., 1984, Electromagnetic Interactions of Molecules With Metal Surfaces, North-Holland Physics Publishing Amsterdam, 113: 195-287; Chance, R. R., Prock, A., and Silbey, R., 1978, Molecular fluorescence and energy transfer near interfaces, Adv. Chem. Phys., 37: 1-65; Barnes, W. L., 1998, Fluorescence near interfaces: the role of photonic mode density, J. Modern Optics, 45(4): 661-699). This theory is complex, mostly focused on thick metal films or mirrors, and difficult to correlate with experimental expectations. There appears to be three types of interactions of fluorophores with smooth metal surfaces, quenching, SPCE, and free space emission. (Ford, G. W., and Weber, W. B. (1984), supra). For example, in a comparison of the relative decay rates at a distance d from a silver surface (Γ(d)) as compared to the rate for the same process in free space (Γ(∞)) at infinite distance from the metal, if the fluorophore is close to the surface (d<20 nm) there is a high rate for radiationless deactivation and the emission is quenched. The silver surface has modest effects on the radiative decay rate at distances up to 200 nm. Depending on the orientation, the reflected field can increase or decrease the rate of emission. At distances from 20-100 nm the dominant decay rate, for the perpendicular dipole, is into the surface plasmon. For a parallel dipole closer than 100 nm the radiative rate is decreased because the oscillatory charge in the fluorophore is partially cancelled by opposite charges on the metal surface. For perpendicular dipoles the oscillating charge on the fluorophore and in the metal create dipoles with the same orientation. The net dipole is increased and the radiative rate increases.

Considering the fraction of the potential fluorescence which results in SPCE, we see SPCE should be observable for any fluorophores which are not quenched, typically those at least 1 nm from the metal surface. In the range from about 10 to 500 nm, decay will occur by both plasmon coupling and emission. A useful range for positioning fluorophores near a conductive surface for SPCE is about 5 to 500 nm, more narrowly 20-500 nm, and more narrowly 20-200 nm (e.g., ±2 nm at the low end and ±10 nm at the high end).

It is interesting to examine the relative probability of the fluorophore decaying by each quenching, free space emission or coupling to surface plasmons. At distances below 20 nm quenching is most probable and above 500 nm the free space emission is dominant. At a distance near 200 nm (≈λ/2) the parallel dipoles show increased free space emission and the perpendicular dipoles show decreased free space emission. This distance dependence results in the oscillatory behavior reported for fluorophores in front of mirrors (Drexhage, K. H., 1970, Influence of a dielectric interface on fluorescence decay time, J. Luminescence, 1, 2: 693-701).

SPCE occurs at distances beyond the range of quenching but close enough to the mirror for the dipoles to interact with the metal. The maximum amount of SPCE occurs from about 20-200 nm for perpendicular dipoles. Coupling of the parallel dipoles is much weaker than for the perpendicular dipoles. The fact that SPCE occurs over longer distances from the surface than quenching suggests that gold films can also be used to couple emission into the prism. Forster transfer to the gold surface is likely to be minimal at distances of 100-200 nm where SPCE is still efficient. Gold is more inert than silver which may be advantageous for the applications of SPCE.

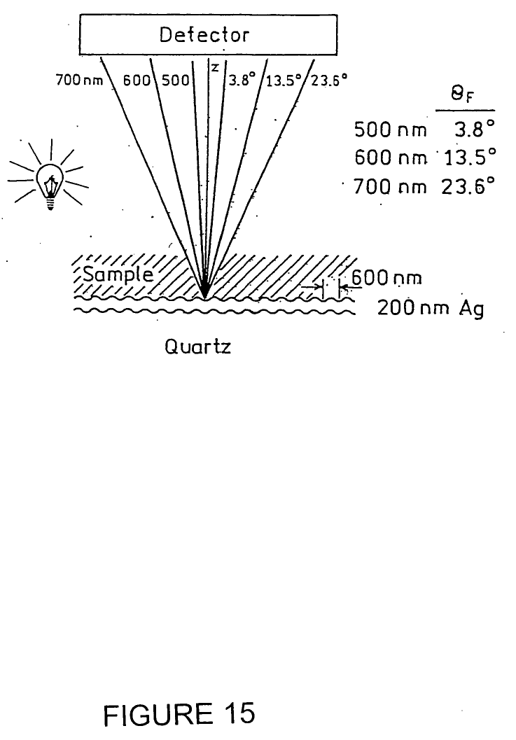

A property of SPCE is a unique dependence on dipole orientation relative to the surface. The orientation dependence for SPCE is opposite than for quenching or emission into free space. That is, SPCE occurs more favorably for perpendicular dipoles. Quenching and emission into free space occur preferentially for parallel dipoles. This suggests that the fabrication of samples with dipoles perpendicular to the surface will result in highly efficient plasmon coupling of the dipoles into the solid medium. The probability of SPCE is highest when the fluorophore is beyond the distance for quenching (>about 20 nm) and closer to the surface than about 500 nm. The interaction between the fluorophores and the metal is a near-field non-radiative interaction. The radiation that couples into the prism is due to the surface plasmon in the metal, not the fluorophore. The decay probability via the plasmons is higher for a dipole perpendicular to the surface, as compared to a parallel dipole. This selectivity for perpendicular dipoles is the result of the required p-polarization for SPR. Dipoles oriented perpendicular to the surface have an electric field with p-polarization. Coupling to the surface occurs over a large range of distances, 20-500 nm (FIG. 15), which is the depth of the evanescent wave in SPRA. This is important for the applications of SPCE because coupling will occur over a significant volume in the sample allowing detection of lower overall analyte concentrations.

Relatively little information is available about the non-radiative pathways where the fluorophore itself is not radiating. Fluorophores are known to be quenched when placed on metallic surfaces (Cnossen, G., Drabe, K. E., and Wiersma, D. A., 1993, Fluorescence properties of submonolayers of rhodamine 6G in front of a mirror, J. Chem. Phys., 98: 5276-5281; Shu, Q. Q., and Hansma, P. K., 2001, Fluorescent apparent quantum yields for excited molecules near dielectric interfaces, Thin Solid Films, 384: 76-84; Campion, A., Gallo, A. R., Harris, C. B., Robota, H. J., and Whitmore, P. M., 1980, Electronic energy transfer to metal surfaces: A test of classical image dipole theory at short distances, Chem. Phys. Letts., 73(3): 447-450; Daffertshofer, M., Port, H, and Wolf, H. C., 1995, Fluorescence quenching of ultrathin anthracene films by dielectric and metallic substrates, Chem. Phys., 200: 225-233), but it is difficult to identify mechanisms without observable emission. Little information is available about coupling to the surface plasmons in thick metal samples because there is no way to observe the plasmons in the opaque metal. Recall that a surface plasmon cannot be excited from the low refractive index side because of an inability to match the surface wavevectors. Similarly, the SP modes of a thick metal cannot emit into the media. Hence any energy which couples from a fluorophore to a thick metal is dissipated by non-radiative processes.

Surface plasmon coupled emission can be observed using the Kretschmann configuration and a prism to couple the excited fluorophore into a plasmon and then into radiation. Several experimental papers have appeared on this topic (Weber, W. H, and Eagen, C. F., 1979, Energy transfer from an excited dye molecule to the surface plasmons of an adjacent metal, Optics Letts., 4(8): 236-238; Benner, R. E., Dornhaus, R., and Chang, R. K., 1979, Angular emission profiles of dye molecules excited by surface plasmon waves at a metal surface, Optics Commun., 30(2): 145-149; Pockhand, I., Brillante, A., and Mobius, D., 1981, Nonradiative decay of molecular excitation at a metal interface, Nuovo Cimento, 63B: 350-357; Eagen, C. F., Weber, W. H., McCarthy, C. F., and Terhune, R. W., 1980, Time-dependent decay of surface-plasmon-coupled molecular fluorescence, Chem. Phys. Letts., 75(2): 274-277; Holland, W. R., and Hall, D. G., 1985, Waveguide mode enhancement of molecular fluorescence, Optics Letts., 10(8): 414-416; Shu, Q. Q., and Hansma, P. K., 2001, Fluorescent apparent quantum yields for excited molecules near dielectric interfaces, Thin Solid Films, 384: 76-84). These papers show that emission can couple through a thin silver surface into the prism. Some of these papers show that the emission occurs only at the surface plasmon angle, with the earliest report appearing in 1975 (Gerbshtein, Yu. M., Merkulov, I. A., and Mirlin, D. N., 1975, Transfer of luminescence-center energy to surface plasmons, JETP Letters., 22: 35-36). One of these experiments was described in an earlier article on Radiative Decay Engineering (Lakowicz, J. R. (2001), supra), which showed angle-dependent emission from rhodamine near a silver-coated hemicylinder (Benner, R. E., Dornhaus, R., and Chang, R. K. (1979), supra). It is instructive to describe this early report on SPCE which appeared in 1979. In these experiments emission was observed using photographic plates to measure the angular distributions of the intensities. A rhodamine solution was excited through a prism at θ_{SP}. The emission was then passed through a filter to select the wavelength, and imaged on photographic plates. A spot towards the left is due to reflected 514 nm light which passed through filters that did not completely remove the incident wavelength. Without a wavelength-selective filter, there was a wide angular distribution of the emission. The angular distribution is considerably more narrow when the emission is passed through narrow band filters. The emission appears at a different location (angle) for each emission wavelength. The presence of different wavelength angles in the top panel was not evident because of the use of black-and-white film. This result demonstrated the existence of SPCE and confirmed the possibility of intrinsic spectral resolution with SPCE.

Many applications of fluorescence technology depend on high sensitivity, so it is useful to determine the efficiency of coupling into the plasmons. Calculations of the probability of coupling to the surface plasmons for fluorophores at various distances above a silver film (Weber, W. H, and Eagen, C. F. (1979), supra) do not reveal what fraction of the coupled energy will appear as far field radiation. However, since SPCE through thin films occurs (Benner, R. E., Dornhaus, R., and Chang, R. K. (1979), supra), the coupling efficiency should indicate the potential collection efficiencies of SPCE. Remarkably, a dipole perpendicular to the surface and 120 nm from the surface may have a 93% probability of coupling to the plasmons. Parallel dipoles couple more weakly with the surface plasmons and this coupling occurs at shorter distances near 200 nm. When averaged over all orientations the coupling efficiency can be over 60% for randomly oriented fluorophores 20 nm above from the surface. This suggests the possibility of capturing over 50% of the emission with simple optical configurations. Efficient light collection can result in considerably increased sensitivity. A conventional fluorescence experiment may use a 1 inch lens 3 or more inches from the sample. For isotropic emission this lens would collect about 1% of the light. As will be shown below, SPCE can be used in geometries that direct essentially all the light to a detector. Hence, the use of SPCE as described herein can result in a 50-fold increase in collection efficiency and sensitivity. If the sample is excited by the evanescent field, then the effective illumination intensity is further increased 10-40 fold, providing an overall increase of up to 1000-fold for SPCE.

Applications of Surface Plasmon-Coupled Emission

There are numerous potential applications of SPCE, particularly in biotechnology, environmental and medical sensing. The experimental configurations useful for SPCE can be very similar to that used for SPRA. These SPRA instruments use thin gold films, scan the angle of incidence, and measure the angle-dependent reflectivity. That approach is similar to measurement of the angle-dependent plasmon-coupled emission. The angular resolution of SPRA devices is far greater than needed for SPCE. If a SPRA instrument scans the angle of incidence, than for SPCE, the excitation and detection channels may be exchanged to allow for excitation at θ_{SP} scanning the observation angle. Hence it appears that SPCE could become an additional or alternative mode of operation for SPRA instruments.

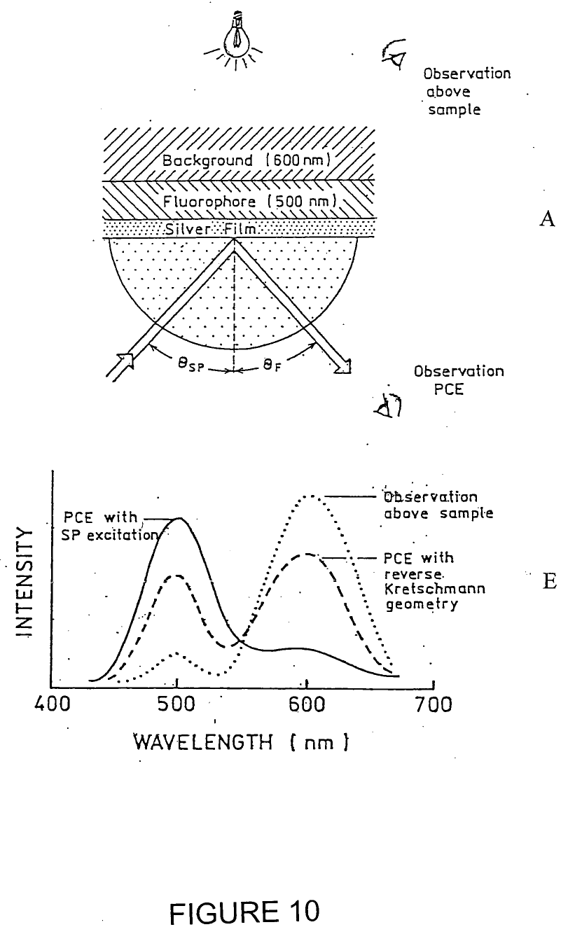

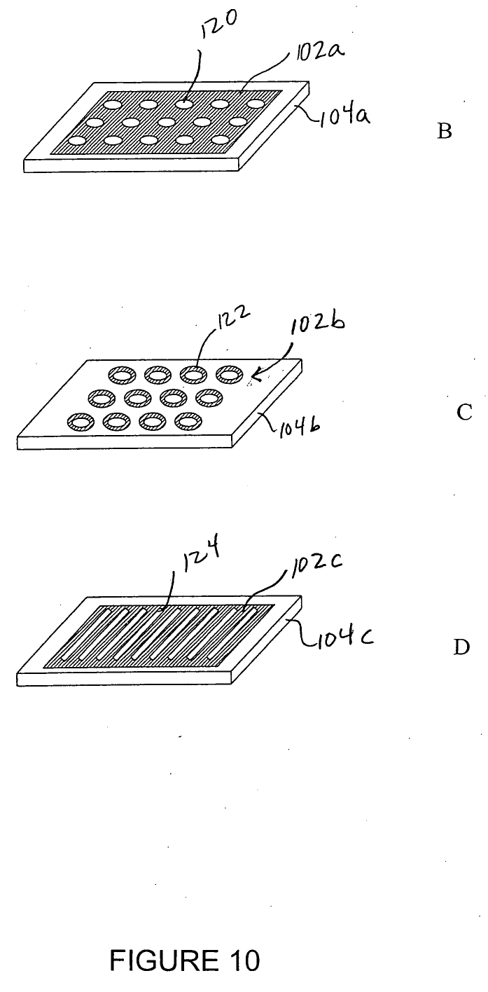

Some potential advantages and configurations for applications of SPCE will now be described. In one exemplary embodiment, there is provided an apparatus for detecting fluorescence in biochemical assays using surface plasmon-coupled emission. FIG. 6 illustrates an exemplary apparatus 100, as does FIG. 10A. Referring to FIG. 6, the apparatus 100 comprises a first layer of conductive material 102 arranged on a first medium 104. The first medium has a first index of refraction and is a solid medium. The first layer of conductive material 102 is situated at an interface between the first medium and a second medium 106. The second medium 106 has a second index of refraction different from the first index of refraction. The first layer 102 can comprise a metal, such as any suitable metal or alloy that is sufficiently inert to the second medium, e.g., a chemical solution. The metal can be deposited onto the first medium by vapor deposition, electroless plating, chemical vapor deposition, photoreduction, or any suitable method known to those of ordinary skill in the art. For example, the first layer 102 can comprise silver, gold, aluminum, or copper, but is not limited to these examples. The first medium can comprise a glass plate, a silica substrate, a polymer substrate, or a prism made of one or more such materials, for example, that is sufficiently transparent to wavelength(s) of light emitted by fluorophore molecules, but is not limited to these examples. The second medium can comprise an aqueous solution, a polymer, or air, for example, but is not limited to these examples. The first layer of conductive material 102 can have a variety of thicknesses and generally should be thin enough so as not to attenuate transmission of the SPCE emission from fluorophores beyond an acceptable level, which can be determined by one of ordinary skill in the art. Attenuation as a function of thickness also depends on the type of material used (e.g., heavier elements tend to attenuate light transmission more than lighter elements), and this aspect can be considered in choosing an appropriate thickness. Generally, thicknesses of 20-100 nm can be used, and more preferably 20-50 nm. Silver films 50 nm in thickness is a useful configuration.

A second layer 108 comprising functional molecules is disposed on the first layer 102. In this example, the functional molecules comprise nucleic acid molecules or polypeptide molecules, or a combination thereof. The functional molecules comprise one or more types of fluorophores and/or are capable of binding analyte molecules comprising one or more types of fluorophores. As used herein, it should be understood that a fluorophore is intended to mean any suitable light emitting molecule, particle, or cluster, including, but not limited to, fluorscein and other conventional light emitting molecules known in the art, semiconductor particles and clusters that can emit light in narrow wavelength bands, e.g., GaAs, and other luminescent nanoparticles. It is advantageous for the second layer 108 to position the fluorophores (e.g., by binding to the fluorophores) within about 5 to 500 nm of the first layer 102. It can also be beneficial for the second layer 108 to position the fluorophores within about 20 to 200 nm of the first layer 102.

The apparatus 100 also comprises an excitation source 112 capable of exciting fluorophores 110 positioned adjacent to the first layer of conductive material 102. The excitation source 112 can comprise a light source capable of producing light comprising an excitation wavelength of fluorophores, the light source being arranged to direct light from the light source toward the first layer 102, such as illustrated in the exemplary embodiment illustrated in FIG. 6. The light source can comprise, for example, a white-light source with suitable filters (e.g., diffraction optics and a selection slit), an ultraviolet lamp, a laser such as a semiconductor laser or other type of laser, a light emitting diode (LED), or any suitable source of light having a suitable wavelength emission to excite fluorophores of interest. Identifying a suitable excitation wavelength and choosing a suitable light source is within the capability of one of ordinary skill in the art.

As illustrated in FIG. 6, the light source can be arranged to direct light comprising the excitation wavelength through the second medium 106 and then to the first layer 102, as illustrated by source 112. Alternatively, the light source can arranged to direct light comprising the excitation wavelength through the first medium 104 and then to the first layer 102, such as illustrated by source 112′. In this case, the angle of incidence on the first layer 102 can be equal to the surface plasmon angle of the excitation wavelength. Also, the second layer 108 can be configured to position fluorophores 110 within an evanescent field at the first layer 102, wherein the evanescent field is generated by light from the light source.

Alternatively, the excitation source can be a source other than light sources as described above. For example, the excitation source can comprise molecules arranged near the first layer of conductive material 102 that exhibit chemiluminescence (CL), bioluminescence (BL), or electrochemiluminescence (ECL). For example, the enzyme molecules for CL or BL could be localized near the first layer 102, resulting in high collection efficiency for the generated emission. Similarly, for ECL, the first layer 12 could be used to both initiate the ECL reaction and couple the emission into the first medium 104. Of course, such electrodes would have to display adequate chemical stability or be coated to protect the surfaces.

The apparatus 100 also comprises a light detector 114 arranged to selectively detect emitted light that is generated by excited fluorophores 110. The detector is arranged to collect emitted light from a predetermined angular range relative to a surface of the first layer 102. The emitted light emanates from the first layer 102 at the surface plasmon angle for an emission wavelength of the excited fluorophores relative to a surface of said first layer 102 and passes through the first medium 104 before being detected by the detector 114. The predetermined angular range over which emitted light is collected comprises the surface plasmon angle for the emission wavelength of the excited fluorophores. The exact mechanism by which light is generated through the coupling of fluorophores to the layer of conductive material is unknown. It may be that light is emitted by the flourophores and passes through the layer of conductive material, or it may be that the fluorophores couple energy to the layer of conductive material by some process and that the light is emitted by the layer of conductive material. The phrase “light generated by fluorophores”, or like phrases, in connection with flourophore emission via SPCE as disclosed herein is not intended to encompass both of these possible explanations as well as other possible explantations, and should not be considered limiting in terms of the exact nature of the physical process.

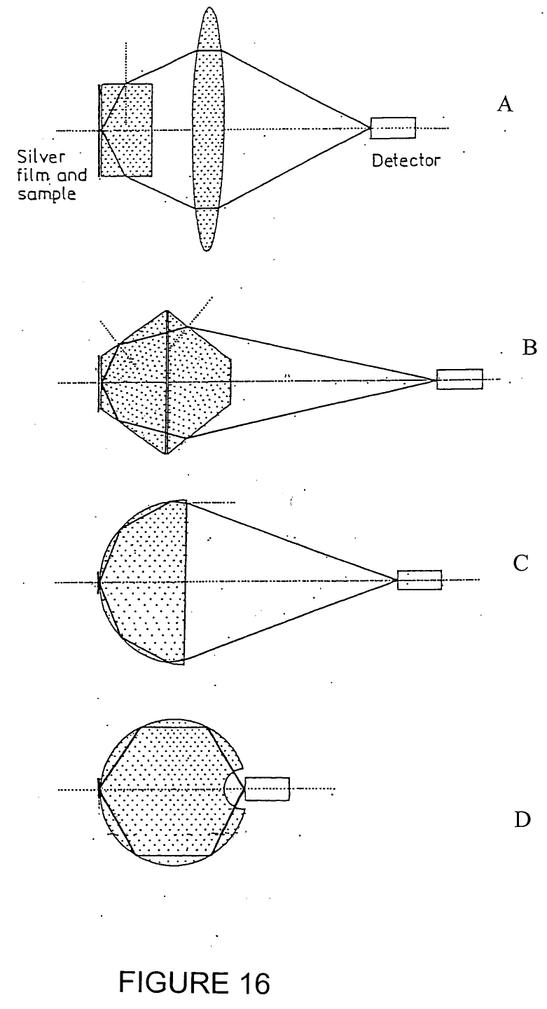

As described previously, SPCE emission emanates from a sample in a conical fashion, that is, along a narrow angular range that corresponds to the shell of a hollow cone. Thus, in one example, the detector 114 can be one that is placed to intercept a portion of that cone. In another example, the apparatus can comprise a focusing element that receives a hollow cone of light emitted by the flourophores and that focuses a portion of the hollow cone of light onto the detector 114, such as shown, for example, in the exemplary illustrations in FIG. 16. For example, the focusing element can comprise a lens situated between said first medium 104 and the detector 114 (FIG. 16, top). In another example, the focusing element can comprise a prism that is capable of redirecting light emitted at the thin layer of conductive material by total internal reflection, such as shown in the two middle panels of FIG. 16. The focusing element can form all or part of said first medium 104, such as illustrated in FIG. 16 (bottom three panels) and FIG. 6. The focusing element can have a shape selected from among polygonal, hemispherical, and spherical shapes, such as shown in FIG. 16, for example.

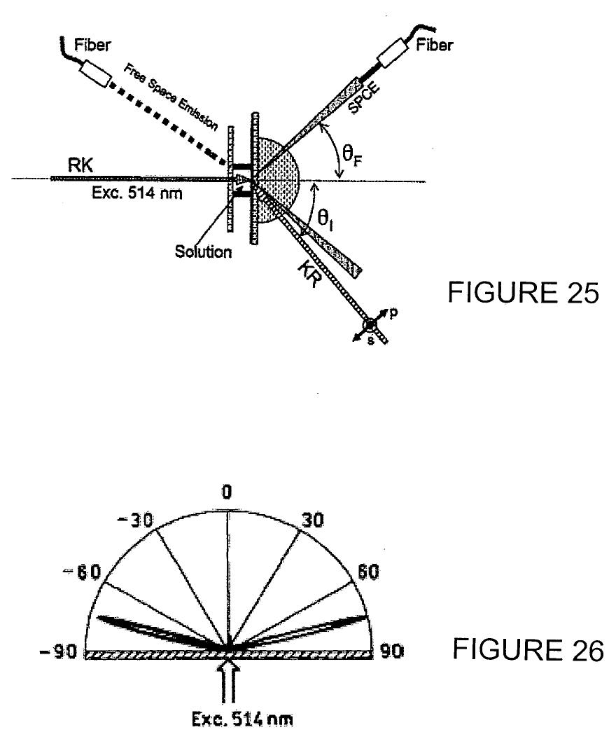

In another aspect, the detector 114 can be an area detector (e.g., a CCD detector) with an area sufficiently large to capture a portion or all of the conical emission, such as illustrated in FIG. 15, for example. The detector 114 can comprise, for example, a photomultiplier tube (PMT), a photodiode, a CCD, or spectrofluorometer, as well as an optional fiber bundle coupled to such a device and positioned to collect the directional SPCE emission, such as illustrated, for example, in FIGS. 25 and 32. The detector 114 can also comprise suitable electronics to generate an electrical signal corresponding to the intensity of light collected, and optionally further corresponding the position of the light detected. Suitable electronics (e.g., personal computer and/or other hardware devices such as amplifiers) can be coupled to the detector for processing measured data.

In another aspect, the apparatus 100 can further comprise a third layer 114 (e.g., a spacer layer) arranged between the first layer of conductive material 102 and the second layer 108 of functional molecules, the third layer comprising at least one of silica, polymer material, protein molecules or lipid molecules. In FIG. 6, the third layer 116 is illustrated as the intersection line between the first layer 102 and the second layer 108. It should be noted that descriptions herein of one layer being disposed on another layer do not preclude the presence of additional intervening layers therebetween.

A benefit of SPCE is suppression of background emission. Consider a sample in which the fluorophores of interest are located within 200 nm of the metal surface, such as in a configuration like that illustrated, for example, in FIG. 10A. In one aspect this localization can be accomplished by adsorbing or conjugating biomolecules to the surface. The sample can be excited in the reverse Kretschmann configuration, that is, from above in the exemplary configuration shown in FIG. 10A. In such a configuration the fluorophores are excited almost uniformly through the thickness of the sample. If the emission is observed from above the sample then the 600 nm background will dominate the emission (lower panel). If the plasmon-coupled emission is observed the signal will be enriched for the 500 nm emission of interest because only fluorophores near the metal will couple efficiently into the prism. More selective observation of fluorophores near the metal surface can be accomplished using the Kretschmann configuration. If the sample is excited through the prism at θ_{SP} then the fluorophores of interest can be selectively excited by the evanescent field which penetrates about λ/2 into the sample, further increasing the desired 500 nm emission and suppressing the background 600 nm emission. The evanescent field is increased about 40-fold relative to the incident field which will further increase the selective observation of fluorophores near the metal surface. Thus, an intensity of the emitted light at the surface plasmon angle from fluorophores adjacent to the first layer of conductive material 102 is enhanced relative to emission from fluorophores located distant from said first layer of conductive material 102, thereby effectively suppressing detection of background emission relative to detection of the emitted light from fluorophores adjacent to the first layer of conductive material 102.

In another aspect, the apparatus 100 can be modified to comprise a glass prism and a glass plate coated with the first layer of conductive material on a side of the plate facing away from the prism, wherein an index matching fluid having substantially the same index of refraction as the glass prism and the glass plate is disposed between the glass prism and glass plate. Such an exemplary arrangement of the glass prism, glass plate and index matching fluid is illustrated in FIG. 1.

In another aspect, the light source 112 can be configured to illuminate a selected region of the second layer 108, and the apparatus 100 can further comprise a time-domain recorder coupled to the detector 114 to record a signal from said detector as a function of time. The signal corresponds to light generated by fluorophores at the selected region. Such an approach provides a way to measure time dependent correlation effects between molecules in the selected region in conjunction with the benefits of SPCE as described herein.

In another aspect, the first layer of conductive material can comprise a patterned structure, such as illustrated in FIGS. 10B-10D. This aspect can be provided in the apparatus 100 and can also be used more generally in such an apparatus without a second functional layer 108, if desired. FIG. 10B illustrates an exemplary embodiment wherein a first layer of conductive material 102a is disposed on a first medium 104a (e.g., a glass, silica or polymer substrate) and comprises a plurality of apertures 120 arranged therein. The apertures 120 can have a substantially uniform size, for example, and can be arranged in a predetermined pattern, such as two-dimensional pattern in the form of a square array pattern, a triangular array pattern, a rectangular array pattern, a hexagonal array pattern, or other suitable pattern. FIG. 10D illustrates an exemplary embodiment of another patterned structure, wherein the first layer 102c is disposed on a first medium 104c and comprises a plurality of apertures 124 therein. The patterned structures illustrated in FIGS. 10B and 10D can both be considered grating structures.

Also, FIG. 10C illustrates an exemplary embodiment of another patterned structure wherein a first layer of conductive material 102b is disposed on a first medium 104b and comprises a plurality of islands 122 of conductive material, such as ring-shaped regions, arranged in a predetermined pattern, such as a two dimensional pattern. Ring-shaped regions in this regard are intended to mean annular structures of any suitable shape (e.g., circles, squares, triangles, rectangles, polygons, etc.) with or without pointed intersection points, and are not limited to circular structures.

The use of patterned structures as described herein can be beneficial because such structures can have light filtering properties. Such light filtering properties of patterned films (or more generally, photonic structures) are known to those of ordinary skill in the art. By using patterned layers of conductive material as described herein, the patterned layers can be designed to transmit light of wavelengths of SPCE emission from fluorophores of interest and to attenuate light of other wavelengths. Thus, such patterned layers can provide for further suppression of background emission and background noise. Of course, as with other embodiments described herein, separate filters can be used to filter light entering detectors to filter out or attenuate light of wavelengths other than wavelengths of SPCE emission from fluorophores. The wavelength (or range thereof) that can pass through a patterned conductive film depends upon both the aperture size (e.g., diameter) and the spacing between apertures as known to those in the art. Selection of appropriate aperture sizes and spacings to achieve a desired filtering characteristic is within the capability of one of ordinary skill in the art either based on existing mathematical treatments or by empirical approaches (e.g., making test films of various configurations and measuring their properties to then determine appropriate parameters).

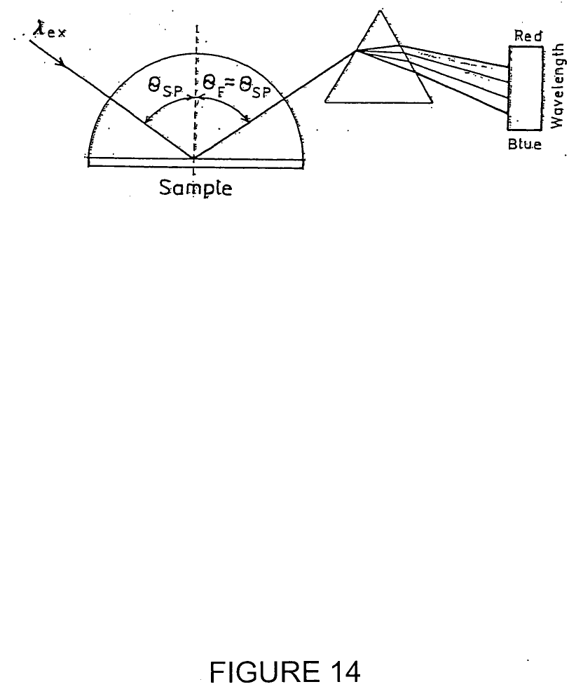

As noted previously in connection with the exemplary configuration shown in FIG. 14, a prism can be used to increase the angular difference between emission wavelengths. This can be useful because the angular separation for various wavelengths for SPCE is modest, about 3° from 500 to 700 nm for silver (e.g., FIG. 3). It should also be noted that the use of patterned structures acting as gratings can also be used to provide larger angular differences across typical emission spectra. For example, periodic silver surfaces could be used since such surfaces can facilitate emission by nearby fluorophores. Fluorescence has been observed from fluorophores directly on or close to silver gratings (Sullivan, K. G., King, O., Sigg, C., and Hall, D. G., 1994, Directional, enhanced fluorescence from molecules near a periodic surface, Appl. Optics, 33(13): 2447-2454; Kitson, S. C., Barnes, W. L., and Sambles, J. R., 1996, Photoluminescence from dye molecules on silver gratings, Optics Commun., 122: 147-154; Knoll, W., Philpott, M. R., and Swalen, J. D., 1981, Emission of light from Ag metal gratings coated with dye monolayer assemblies, J. Chem. Phys., 75(10): 4795-4799; Amos, R. M., and Barnes, W. L., 1999, Modification of spontaneous emission lifetimes in the presence of corrugated metallic surfaces, Phys. Rev. B., 59(11): 7708-7714). The silver surfaces were about 200 nm thick, and the grating was opaque. The periodic structure on the grating provides another mechanism to match the need for wavevector matching at the interface, making it possible to excite plasmon in metallic gratings from air. In fact, such plasmons are the origin of the Wood's anomaly in which gratings become non-reflective at a particular wavelength. Since the periodic structure provides a mechanism for coupling incident light into surface plasmons, it should also provide a mechanism for coupling the energy of the excited fluorophores into radiation.

Suppose the sample is on a opaque silver grating with a pitch of λ_{G}=600 nm. The condition for plasmon resonance is now given by
k₀ sin θ_{SP}^{G}=±k₀ sin θ_{SP}^{M}+nG (15)
where n is an interger and G is the propagation constant of the grating,

$$G = \frac{2 π}{{λ}_{G}} .$$

The superscripts G and M refer to a grating and planar surface, respectively. Recalling that

$${K}_{0} = \frac{2 π}{{λ}_{0}}$$

this expression can be rewritten as

$$sin {θ}_{SP}^{G} = \pm sin {θ}_{SP}^{M} \pm n \frac{{λ}_{0}}{{λ}_{G}} \qquad ( 15 )$$

The presence of the additional term has a dramatic effect on the angles for coupling to the plasmon to different wavelengths. For example, consider a grating of silver and assume that θ_{SP} for a planar surface is 50°. One can readily calculate that θ_{SP} for 500 nm is now near 20 (e.g., FIG. 15). Emission at 600 and 700 nm can appear at near 16° and 28° from the normal, respectively. Hence one could readily collect an emission spectrum comprising multiple wavelengths with an array detector (e.g., a CCD detector) such as shown, for example, in FIG. 15.

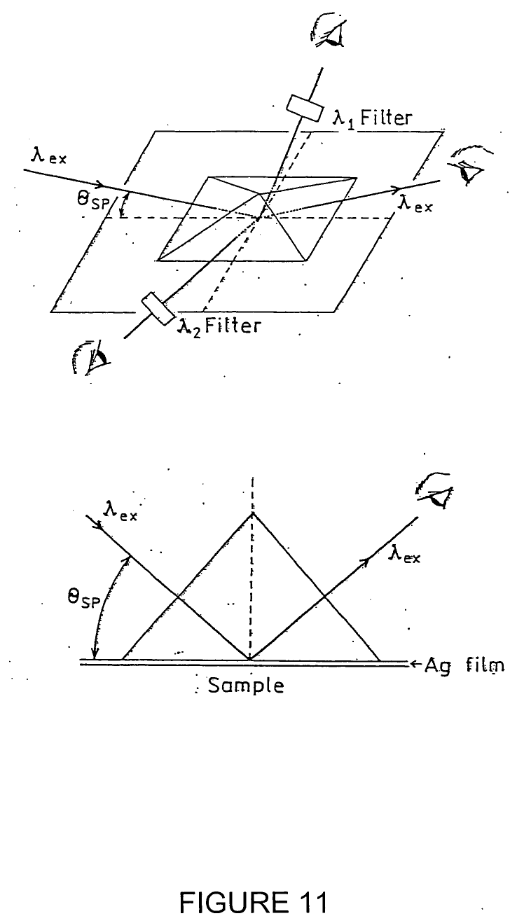

Another approach to using SPCE is to take advantage of the intrinsic angular shifts for different wavelengths. This property of SPCE can be used to provide approaches to wavelength-ratiometric measurements. For example, a triangular prism can be coupled to a sample having a silver film such as shown in FIG. 11. The sample can be excited at θ_{SP} to obtain an enhanced localized field in the sample. The intensity of the excitation source or the reflectivity could be observed on the opposite side in the plane of incidence. Two additional detectors can be positioned at the two 90° angles and the desired wavelengths selected with filters. This configuration would provide simultaneous measurement of three signals and efficient collection of the emission due to plasmon coupling. The prisms could be as small as needed for the desired application. Multiple prisms could be placed on the substrate for high throughput or multi-analyte measurements. The faces of the pyramids could be shaped to direct the SPCE can be directed towards a detector.

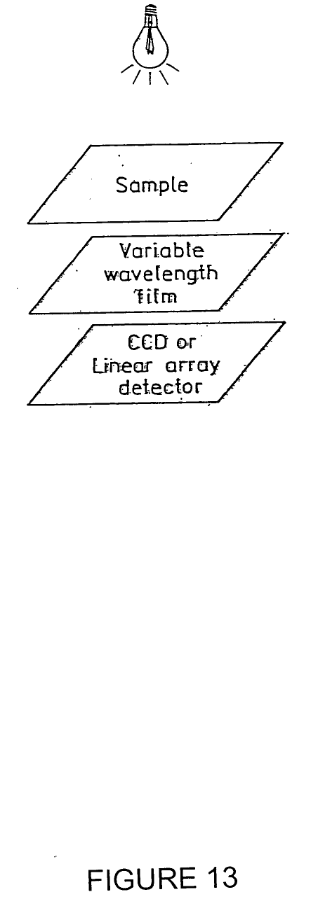

In another aspect, plasmon-coupled emission can also be used to collect emission spectrum simultaneously at multiple wavelengths. In this regard, the exemplary apparatus 100 illustrated in FIG. 6 can have functional molecules that either comprise a plurality of types of fluorophores or are bound to analyte molecules comprising a plurality of types of fluorophores, wherein fluorescence emission of each type of fluorophore has a different emission wavelength, and wherein the detector is configured to selectively detect light generated by each type of fluorophore by collecting light generated by different types of fluorophores at different angles. For example, a large sample area can be illuminated in a reverse Kretschmann configuration such as shown in the exemplary illustration of FIG. 13. The emission could be coupled through a variable-wavelength filter and then to a CCD or linear array detector. These configurations would provide both high efficiency collection and simultaneous observation at multiple wavelengths. While different wavelengths have different coupling angles, the angular shifts are only a couple degrees. The variable wavelength filter needs to be thin to allow proximity focusing of the different wavelengths, or it may be useful to add a translucent sheet to stop lateral migration of the coupled emission. Of course the exemplary configuration shown in FIG. 13 would work with a metal film. In addition, the directional nature of SPCE makes it possible to use dispersive optics without focusing lenses such as shown, for example, in FIG. 14. The SPCE could be passed through a prism, separating the wavelengths, and then observed using a linear array detector. In addition, in connection with multiple types of flourophores that emit multiple wavelengths, the first layer 102 can comprise a patterned structure, such as described above, that provides further angular separation between light generated by different types of fluorophores.

In another aspect, the apparatus 100 can comprise conductive particles having diameters less than about 200 nm (and more narrowly less than about 100 nm±10 nm) disposed on the first layer 102. Such particles, which can be referred to as nanoparticles, can further enhance light emission from fluorophores. Such particles can be prepared using conventional techniques for preparing such particles and clusters, such as solution chemistry and gas condensation in a supersaturated vapor. The particles can comprise, for example, gold, silver or aluminum.

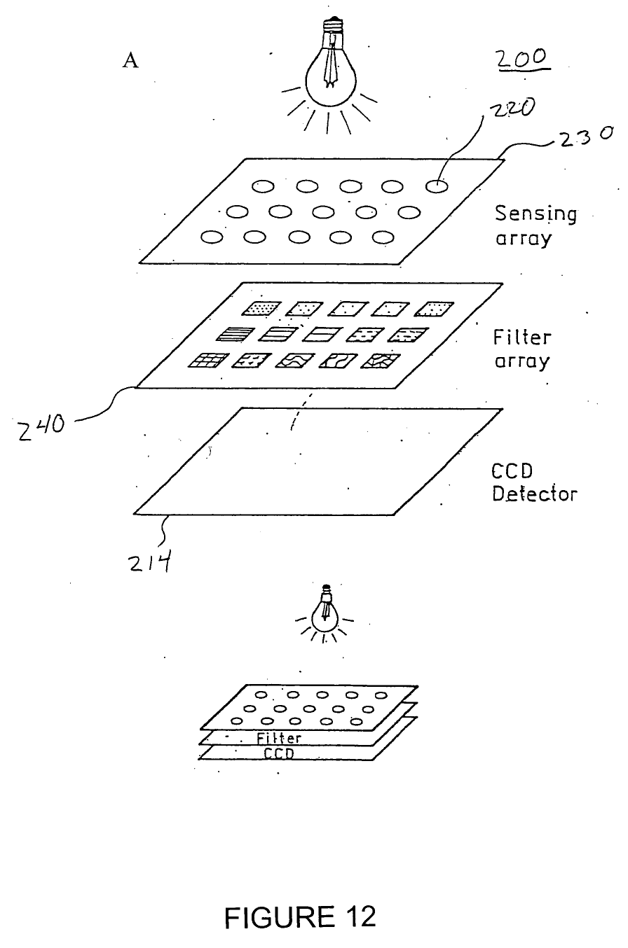

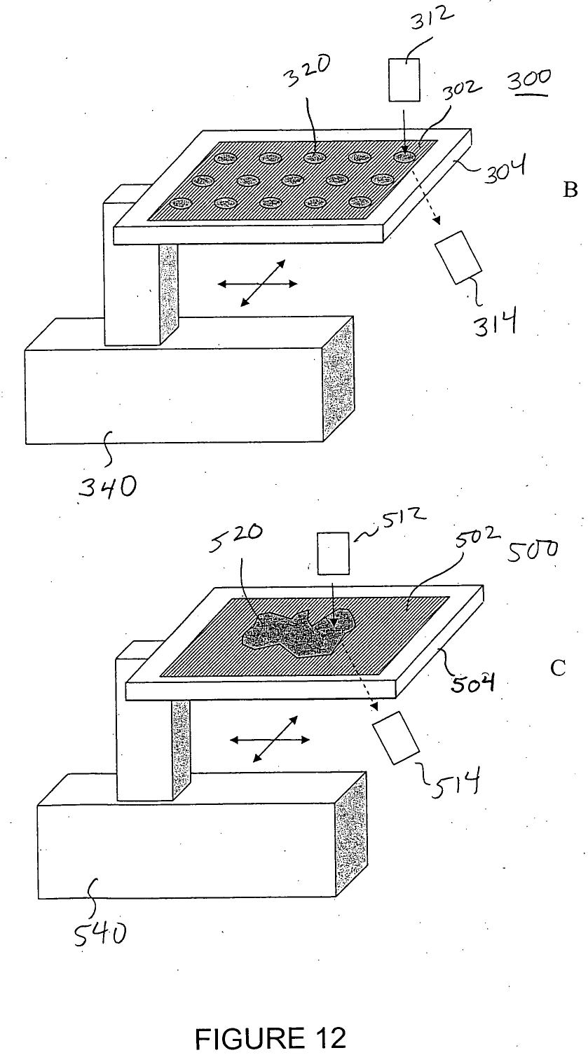

In another aspect, the second layer 108 comprising functional molecules can comprise a plurality of functional regions, the functional regions being separated from one another laterally and being arranged in a predetermined pattern on the first layer, at least some of the plurality of functional regions comprising functional molecules that are different from functional molecules of other ones of the plurality of functional regions. An example is shown in connection with the exemplary apparatus 200 illustrated in FIG. 12A, wherein a plurality of functional regions 220 are disposed on a member 230 that can comprise a layer of conductive material on a first medium such as described above. An optional filter array 240 is also shown. A CCD detector 214 collects light emitted by fluorophores at the functional regions 220. Another example is shown in connection with the exemplary apparatus 300 illustrated in FIG. 12B, wherein a plurality of functional regions 320 are disposed on a layer of conductive material 302 disposed on a first medium 304, which is attached to a translation stage 340 for translating the first medium 304 and functional regions 320 relative to a light source 312 and a detector 314. In either example (FIG. 12A or FIG. 12B), the detector selectively detects light from individual functional regions. As discussed previously a focusing element could also be used in to further focus light on either detector 214 or 314.

As illustrated in FIG. 12B, the apparatus 300 can comprise a mechanism that allows the light source to successively illuminate different positions on the first layer 302 and that allows the detector to detect the light generated by the fluorophores as a function of the illumination of the different positions. The detector thereby generating two-dimensional data from the light generated by the fluorophores. In the example of FIG. 12B, the mechanism is a translation stage 340 that provides relative motion between the first medium and the light source and between the first medium and the detector. However, other mechanisms could be used, such as an optical scanner that scans a light beam over the layer 102 in conjunction with movable or stationary focusing optics that collects light and directs to the detector 314.

Such arrays of functional regions can be generally of the type being used in genomics or proteomics, such as shown, for example, in FIG. 12A. The only required change compared to conventional arrays is the addition of a layer of conductive material such as a thin metal film. The entire array could be illuminated in the reverse Kretschmann configuration. Each spot on the array would couple through the metal, can be observed through different emission filters, and then recorded on a CCD detector. Proximity focusing would result in an efficient and compact device. Additionally, the metal films used for SPCE are highly reflective which would prevent most of the incident light from reaching the detector. The spots may need to be further apart than in high density DNA arrays because of the cone of emission and propagation of plasmon across the surface.

As suggested above, SPCE can be used for high sensitivity detection. Several examples are shown in FIG. 16, which was also discussed above. The most direct approach is to collect the SPCE with an appropriately sized lens. Because of the large angles for SPCE it can be preferable to use two optical elements rather than one larger lens (e.g., FIG. 16, top). Essentially all the emission could be focused on the detector. A single optical element shaped like a hexagon could accomplish the same task using TIR of the coupled emission (e.g., FIG. 16, second from top). Such an element could be simplified further using a hemisphere with the sample located on the spherical surface (e.g., FIG. 16, second from bottom). Also, light collection and focusing onto the detector could be accomplished. e.g., with no free-space optics, using a glass sphere (e.g., FIG. 16, bottom). In all these configurations the excitation can occur from the sample side, outside the optical element, or by the surface plasmon evanescent wave.

In view of the above, it will be apparent that the present invention method provides a method for detecting fluorescence in biochemical assays using surface plasmon-coupled emission. The method comprises arranging an assay device proximate to a light detector, the assay device comprising a first layer of conductive material arranged on a first medium with a second layer comprising functional molecules disposed on the first layer, such as described above.

The method also comprises causing fluorophores to be adjacent to said first layer of said assay device and exciting at least some of said fluorophores with an excitation source. The method further comprises detecting emitted light that is generated by excited fluorophores with a detector wherein the emitted light has an emission wavelength of the fluorophores and emanates from said first layer of conductive material at the surface plasmon angle of the emission wavelength relative to a surface of said first layer, and passes through the first medium before being detected by the detector, such as described above.

The fluorophores can be positioned adjacent to the first layer by applying a coating comprising the fluorophores onto said first layer using any known method for providing such coatings as conventionally known in the art. Alternatively, the fluorophores can be positioned adjacent to the first layer of conductive material by exposing the second layer that comprises functional molecules to analyte molecules that comprise the fluorophores. In this way an assay can be carried out by allowing the analyte molecules to bind to said functional molecules and by detecting emitted light.

The method can further comprise exposing the second layer comprising functional molecules to a plurality of substances comprising a plurality of different types of fluorophores to do multicomponent assays involving multiple wavelengths. The detection of multiple wavelengths from multiple types of fluorophores in this regard can be carried out such as described above. In the method, the analyte molecules can comprise one or more of antibodies, fragments of antibodies, peptide antigens, nucleic acids, and polypeptides, wherein said analyte molecules comprise one or more types of fluorophores. Fluorescence emission of each type of fluorophore can have a different emission wavelength, and emission from each type of fluorophore can selectively detected by collecting light emitted at an angle corresponding to the surface plasmon angle for the emission wavelength of each type of fluorophore, such as described above.

In the method, detecting emitted light can comprise selectively detecting light emitted into said first medium in the form of a hollow cone and that has been directed to the detector by a focusing element, such as described above in connection with FIG. 16. Also, the method can comprise passing the emitted light through a third layer arranged between the first layer and the second layer before detecting the emitted light with the detector, wherein the third layer comprises at least one of silica, polymer material, protein molecules or lipid molecules. Other aspects discussed in connection with various exemplary apparatuses described herein are applicable to the method for detecting fluorescence in biochemical assays.

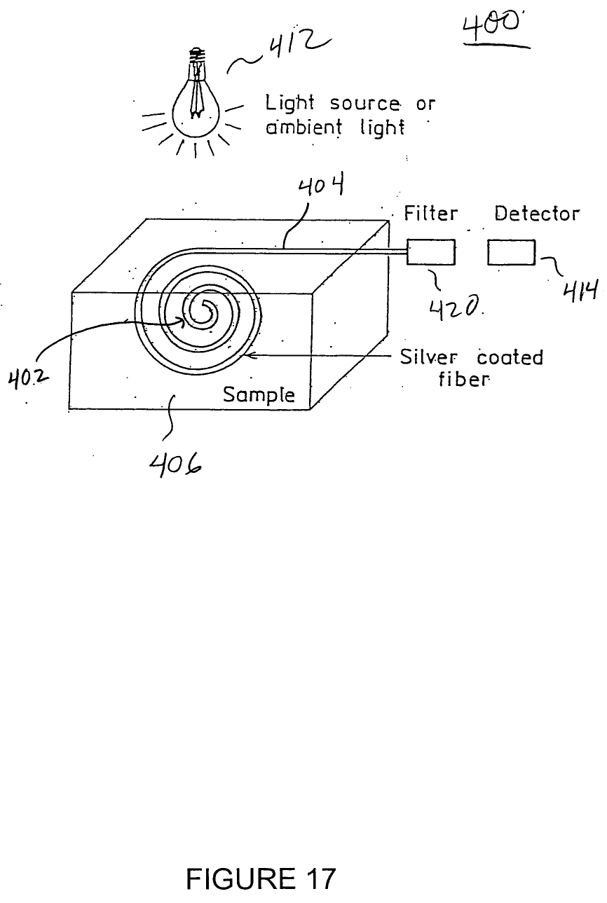

Another approach to obtain high sensitivity can utilize an optical coated with a thin layer of conductive material such as illustrated in FIG. 17. In this regard, FIG. 17 illustrates an exemplary apparatus 400 for observing surface plasmon-coupled emission. The apparatus comprises an optical fiber 404 having a first index of refraction and having a surface portion coated with a first layer of conductive material 402, the first layer of conductive material 402 being situated at an interface between the optical fiber 404 and a medium 406 (sample). The medium 406 has a second index of refraction different from the first index of refraction. The apparatus also comprises a second layer comprising functional molecules disposed on the first layer 402, the functional molecules comprising at least one of nucleic acid molecules and polypeptide molecules, the functional molecules comprising one or more types of fluorophores and/or being capable of binding to analyte molecules comprising one or more types of fluorophores. The apparatus 400 also comprises an excitation source 412, which can be a source of light, capable of exciting fluorophores positioned adjacent to the first layer, and a light detector 414 optically coupled to the optical fiber 404 and arranged to collect emitted light generated by excited fluorophores, wherein the emitted light passes through the optical fiber 404 to the detector 414 and has an emission wavelength of the fluorophores. An optional filter 420 can also be provided to filter or attenuate wavelengths other than SPCE wavelengths associated with fluorophores adjacent to the fist layer 402 of conductive material. The apparatus can employ various materials and variations for the various layers and components such as described in connection with the apparatus 100 of FIG. 6.

An method for observing surface plasmon-coupled emission using such an optical fiber 404 comprises optically coupling 404 optical fiber to the light detector 414, causing fluorophores to be adjacent to said first layer of conductive material 402, exciting at least some of the fluorophores adjacent to said first layer with the excitation source 412, and detecting light generated by excited fluorophores with the detector 414, the emitted light passing through the optical fiber 404 to the detector 414. Certain aspects of the assay method described above in connection with apparatus 100 can also be applied to the method for observing fluorescence with the optical fiber 404. For example, the first layer of conductive material 402 can be formed with metals such as described above, and suitable functional molecules and fluorophores such as described herein can be applied.

In connection with the optical fiber approach, if the surface chemistry is located on the layer of conductive material 402, the desired signal could be obtained even in samples displaying high autofluorescence. Only emission from within λ/2 of the surface of layer 402 (e.g., a metal surface) would be collected by SPCE. Excitation could be accomplished from within the fiber, or from an external source. It is even possible to imagine the use of ambient light for excitation.

In another embodiment, there is provided a method of imaging fluorescence emission from one or more types of fluorophores bound to a cellular sample. An exemplary apparatus 500 for carrying out such imaging is illustrated in FIG. 12C. The method comprises placing a cellular sample 520 on a layer of conductive material 502 disposed on a first medium 504. The cellular sample can be a tissue sample or other biological sample, for example. The first medium 504 has a first index of refraction and is a solid medium. The layer of conductive material 502 is situated at an interface between said first medium 504 and a second medium (e.g., air, aqueous solution, polymer coating, etc.), the second medium having a second index of refraction different from the first index of refraction. The method also comprises exposing the cellular sample to one or more substances capable of binding to one or more types of molecules in said cellular sample, wherein the substances comprising one or more types of fluorophores, thereby causing fluorophores to be adjacent to said layer of conductive material. As with other methods described herein, the substances can be any substance that is desired to be tested for the presence of a given species. The method also comprises illuminating a selected position on the layer of conductive material 502 at an excitation wavelength of said fluorophores, and detecting emitted light that is generated by excited fluorophores at the selected position with a detector 514, tje emitted light having an emission wavelength of the fluorophores. The emitted light emanates from said layer of conductive material at the surface plasmon angle of said emission wavelength relative to a surface of said layer of conductive material and passes through said first medium 504 before being detected by the detector 514. The method further comprises successively illuminating new selected positions on the layer of conductive material 502 and detecting light emitted at each new selected position. This can be accomplished, for example, using a translation stage 540 illustrated in FIG. 12C, or alternatively, using any suitable scanning and deflection optics and suitable collection optics, such as noted above with regard to FIG. 12B.

The signal can be read from the detector 514 using appropriate electronics to thereby generate two-dimensional data as a function of “pixel” location on the sample 520 to provide a two-dimensional image of fluorophore emission associated with SPCE from the sample 520. Various materials and types of components and variations can be used for the first medium 504, the layer 502, the source 512 and the detector 514 such as have been described above in connection with other embodiments. For example, wavelength filtering at the detector can be provided, if desired, to suppress unwanted radiation into the detector, and fiber optics (e.g., fiber bundles) can be used in connection with the detector 514 to collect light.

As noted above, high sensitivity detection can be accomplished with methods which do not result in light-induced background signals, such methods including chemiluminescence (CL) bioluminescence (BL) and electrochemiluminescence (Kricka, L. J., 1988, Clinical and biochemical applications of luciferases and luciferins, Anal. Biochem., 175: 14-21; Akhavan-Tafti, H., Reddy, L. V., Siripurapu, S., Schoenfelner, B. A., Handley, R. S., and Schapp, A. P., 1998, Chemiluminescent detection of DNA in low- and medium-density arrays, Clin. Chem., 44(9): 2065-2066; Leong, M. M. L., and Fox, G. R., 1990, Luminescent detection of immunodot and western blots, Methods in Enzymology, 184: 442-451; Chappelle, E. W., Picciolo, G. L., and Deming, J. W., 1978, Determination of bacterial content in Fluids, Methods in Enzymology, 57: 65-72; Bolton, E., and Richter, M. M., 2001, Light emission at electrodes: An electrochemiluminescence demonstration, J. Chem. Ed. Chem., 78(5): 641-643). Since SPCE does not depend on the mode of excitation, SPCE will also occur for these non-photoluminescent phenomena. For example, the enzymes needed for CL or BL could be localized near the metal film, resulting in high collection efficiency for the generated emission. Similarly, for ECL, the metal film could be used to both initiate the ECL reaction and couple the emission into the prism. Of course the metallic electrodes will have to display adequate chemical stability or be coated to protect the surfaces.

While the invention has been described in detail with reference to exemplary embodiments, it will be apparent to one skilled in the art that various changes can be made, and equivalents employed, without departing from the scope of the invention. The following examples further illustrate principles described above and further illustrate exemplary implementations. However, the methods and implementations of the invention are not limited to the following examples.

### EXAMPLES

### Example 1

### DNA Hybridization Using Surface Plasmon-Coupled Emission

DNA hybridization can be measured using surface plasmon-coupled emission, SPCE. Excited fluorophores couple with surface oscillations of electrons in thin metal films. These surface plasmons then radiate into the glass at a sharply defined angle determined by the emission wavelength and the optical properties of the glass and metal. This radiation has the same spectral profile as the emission spectrum of the fluorophores. The emission due to Cy3-labeled DNA oligomers bound to complementary unlabeled oligomers which were themselves bound to the metal surface was studied. Hybridization resulted in SPCE into the prism due to Cy3-DNA. Directional SPCE was observed whether the sample was illuminated from the sample side or through the glass substrate at the surface plasmon angle for the excitation wavelength. A large fraction of the total potential emission is coupled to the surface plasmons resulting in improved sensitivity. When illuminated through the prism at the surface plasmon angle the sensitivity is increased due to the enhanced intensity of the resonance evanescent field. As described above, SPCE depends on proximity to the metal surface, e.g. silver, gold, aluminum, copper, and the like. As a result, changes in emission intensity are observed due to fluorophore localization even if hybridization does not affect the quantum yield of the fluorophore. The use of SPCE resulted in suppression of interfering emission from a non-complementary Cy5-DNA oligomers due to weaker coupling of the more distant fluorophores with the surface plasmons. SPCE has numerous applications to nucleic acid analysis and for the measurement of bioaffinity reactions.

Materials and Methods

All samples were prepared on quartz slides, which were smooth, ungrooved parts of 1 mm-demountable cuvettes (12.5 mm×45 mm; Stama Cell, Inc., Atascadero, Calif.). Complementary oligonucleotides labeled with biotin or Cy3 (N,N′-(dipropyl)-tetramethylindocarbocyanine) and non-complementary oligo labeled with Cy5 (N,N′-(dipropyl)-tetramethylindodicarbocyanine) were obtained from the Biopolymer Core Facility at the University of Maryland, School of Medicine. Nanopure H₂O (>18.0 MΩ), purified using Millipore Milli-Q Gradient System, was used for all experiments. Buffer components were purchased from Sigma-Aldrich (St. Louis, Mo.).

Each quartz slide was half covered with a continuous 50 nm thick silver film, which was vapor deposited by EMF Corp. (Ithaca, N.Y.). The entire surface was then covered with 500 mL of an aqueous solution of 10 mM BSA-biotin (Sigma, St. Louis, Mo.) and placed in humid chamber for 20 hours, 5° C., After being washed 3 times with water the slides were placed again in humid chamber and 500 mL of 5 mM streptavidin (Molecular Probes, Eugene, Oreg.) in 0.1×PBS buffer was deposited on each BSA-biotin-coated surface for 40 min (room temperature). These slides were than washed 3 times with 0.1×PBS buffer. Then 500 mL of biotinylated DNA samples in 5 mM HEPES (pH 7.5), 0.1 M KCl and 0.25 mM EDTA were deposited for 1 hour at room temperature. After washing with the same buffer, and covering with second part of demountable cuvette, the samples that consisted of ssDNA-streptavidin-BSA layer on the silver film were ready to use.

Determining the conditions needed to observe SPCE was made with the double-stranded Cy3-DNA-biotin deposited on protein monolayer by incubation with 500 mL of 2 mM Cy3-DNA-biotin previously hybridized by mixing complementary oligos in 5 mM HEPES (pH 7.5), 0.1 M KCl and 0.25 mM EDTA.

For hybridization experiments, the protein layer was covered with 500 mL of 2 mM single-stranded biotinylated oligo (ss DNA-biotin) in buffer solution. The 1 mm cuvette was then filled with hybridization buffer containing 5.4 nM complementary ss Cy3-DNA and 150 nM non-complementary ss Cy5-DNA.

Fluorescence Measurements

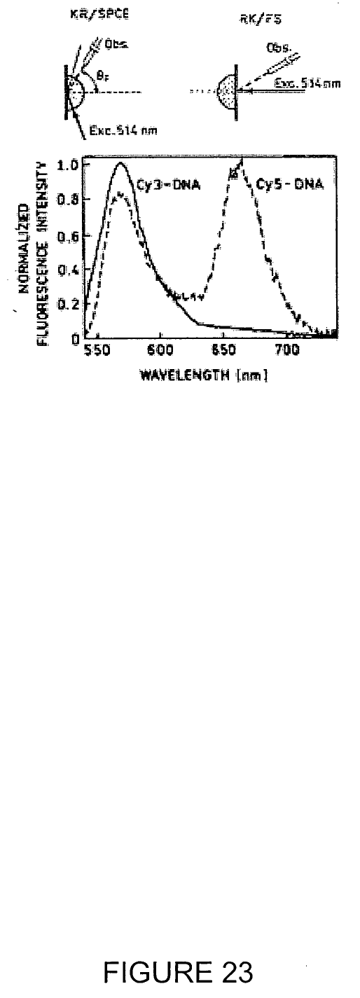

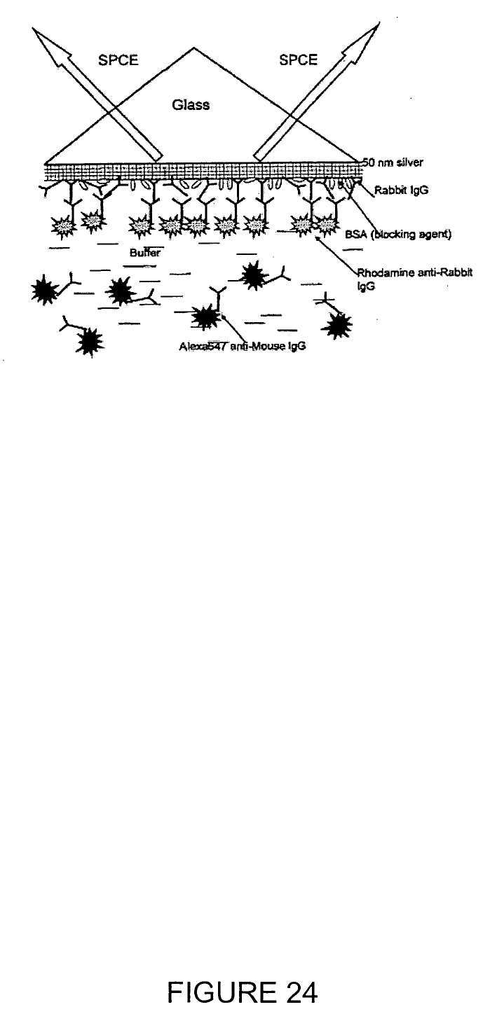

The quartz slide with sample was attached with index-matching fluid to a hemi-cylindrical prism made of BK7 glass and positioned on a precise rotary stage. The stage was equipped with an arm about 15 cm long for fiber optic detection. The fiber bundle was 3 mm in diameter. The input of the fiber optic bundle could be rotated around the prism, which allowed observation at any angle relative to the incident angle. The incident light was either normal to the glass water interface from the water side (reverse Kretschmann, RK) or incident at the SPR angle for the incident wavelength through the prism (Kretschmann configuration, KR). These optical configurations will be described in more detail in the Results section and are shown schematically in FIG. 23. For collection of the angle dependent emission intensity a 200 μm air slit was placed on the fiber input. The output of the fiber was directed to a SLM 8000 single photon counting spectrofluorometer. For measurement of the emission spectra the 200 μm slit was removed from the fiber and the fiber input was positioned as close as possible to the sample. The 514 nm excitation was from a pulsed mode-locked argon ion laser (76 MHz repetition rate, 120 ps half-width). Scattered incident light at 514 nm was suppressed on observation by using a holographic supernotch-plus filter (Kaiser Optical System, Inc., Ann Arbor, Mich.). Unless otherwise indicated the incident light was polarized horizontally in the laboratory axis, that is p-polarized relative to the plane of incidence.

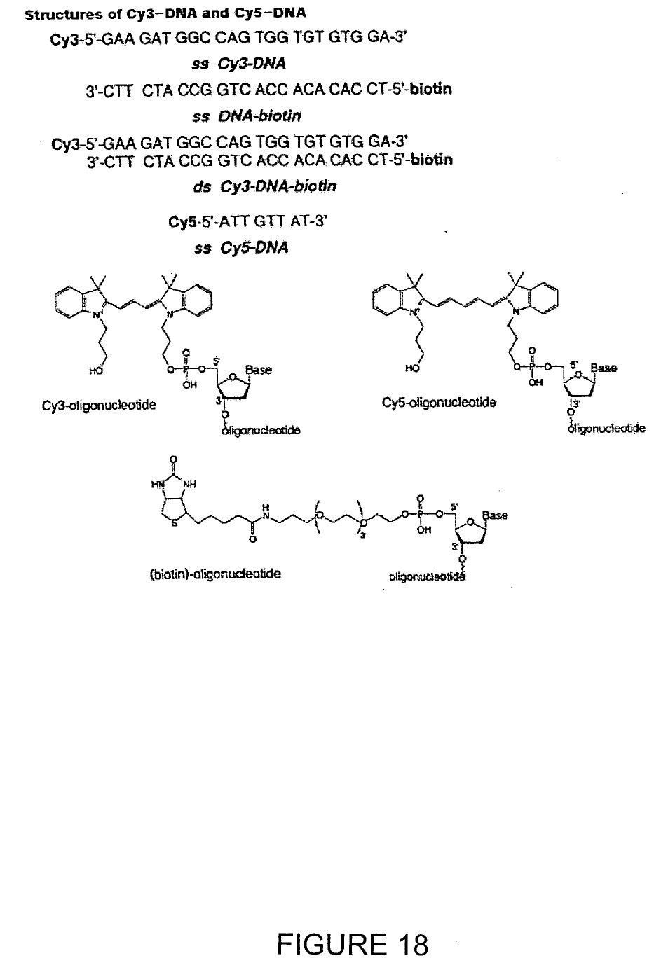

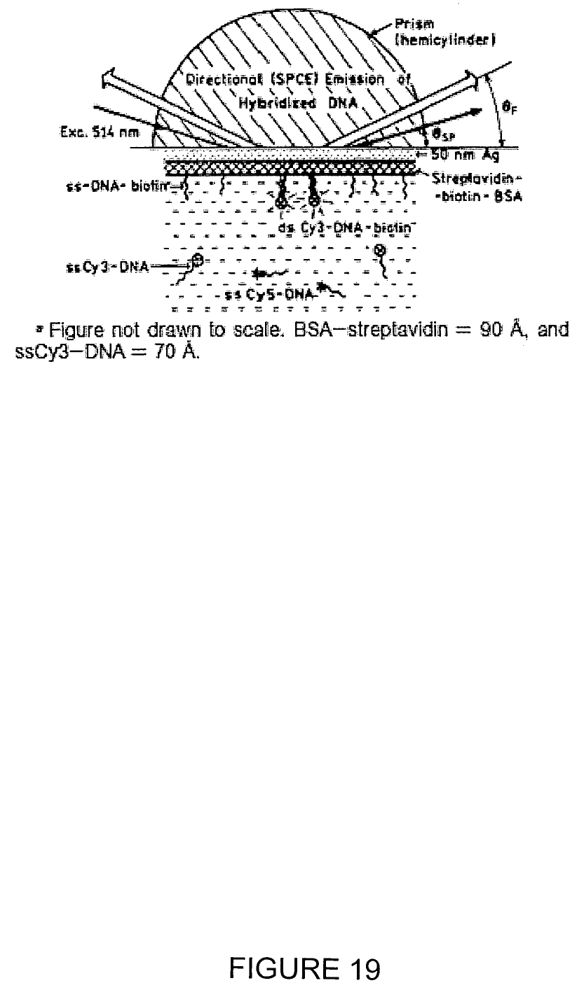

The DNA oligomers are shown in FIG. 18. The surface-bound capture oligomer was labeled with biotin (ssDNA-biotin). The oligomer complementary to the capture oligomer was labeled with Cy3 (ssCy3-DNA). A shorter oligomer which was not complementary to the capture oligomer was labeled with Cy5 (ssCy5-DNA). The arrangement of the experiment is illustrated in FIG. 19. The ssDNA-biotin is bound to the silver surface by a layer of biotinylated BSA covered with streptavidin. ssDNA-biotin binds to this surface. The bathing solution can contain ssDNA-Cy3 and/or ssDNA-Cy5. It was expected that some of the ssDNA-Cy3 to bind to the surface and any excess to remain unbound. ssDNA-Cy5 will be unbound and more distant from the silver.

When using silver surfaces two different modes of excitation are preferred. The sample can be excited through the aqueous phase, in our case with normal incidence to the interface. This is called the reverse Kretschmann (RK) configuration which does not result in excitation of surface plasmons in the metal. A second mode of excitation is through the glass substrate with the incident angle equal to the SPR angle for the excitation wavelength. This is called the Kretschmann configuration (KR), illustrated in FIG. 19. The excitation source is the evanescent field from the plasmon resonance. This field penetrates about a wavelength into the aqueous phase. Because of the resonance interaction the evanescent intensity is enhanced about 20-fold relative to the incident intensity. This evanescent field is different from that found for total internal reflection (TIR) because it is the result of surface plasmons in the metal film. However, the origin is similar because the incident light cannot propagate into the aqueous phase.

Detection of SPCE

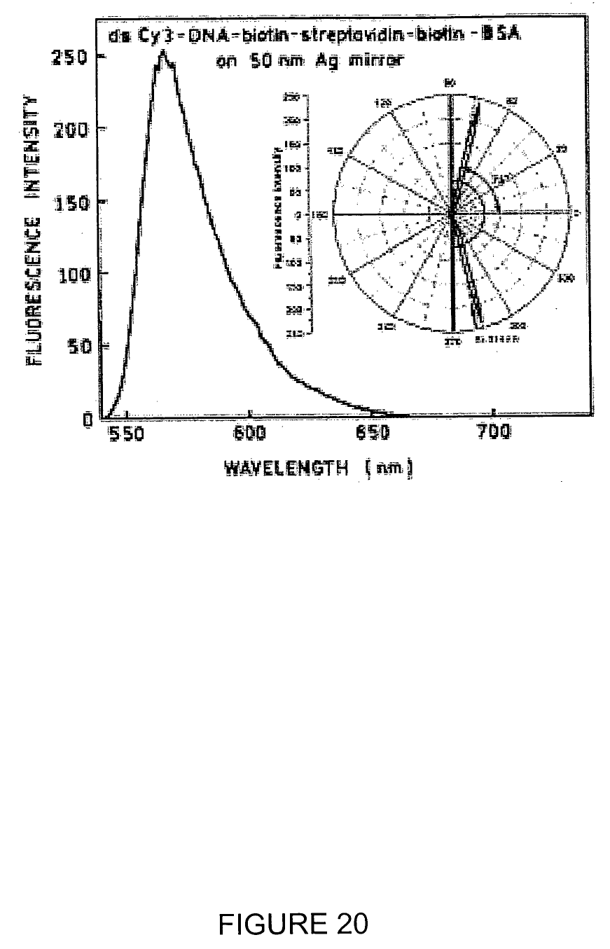

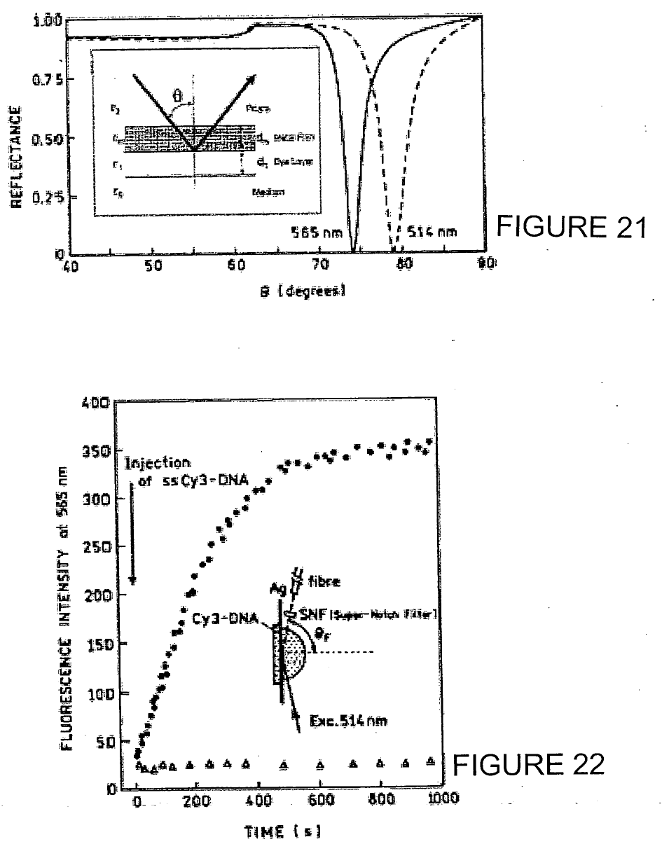

First the sample was examined with surface plasmon (KR) excitation. Because it was desired to measure the angular distribution of the SPCE this sample contained only surface bound Cy3-DNA. The emission intensity was measured at all angles ±90° from the normal axis (FIG. 20). The emission was found to be strongly directional at ±75°. Even though the emission is the result of surface plasmons the emission spectrum is characteristic of Cy3. This angle was compared with the expected angle-dependent reflectivity of the silver film. For this calculation the assumption was made that the protein-DNA layer was 15 nm thick and had a slightly larger dielectric constant (1=2.07) than the aqueous phase (0=1.79). This calculation (FIG. 21) showed a reflectivity minimum near 74° for the emission wavelength of 565 nm, in good agreement with the observed value of 75°. The similarity of the observed and calculated angles, and the highly directional nature of the emission, supports our claim that the emission at 75° is in fact surface plasmon coupled emission. Additionally, the emission was p-polarized as expected for SPCE. The reflectivity was also calculated for the excitation wavelength of 514 nm. The calculated reflectivity minimum agrees with the experimental value of 78° which was found to yield the most intense emission.

DNA Hybridization

The excitation and emission angles were chosen from the results in FIG. 20, and were 78° and 75°, respectively. The sample consisted of unlabeled ssDNA-biotin which was bound to the protein layer. Upon injection of ssCy3-DNA there was a time-dependent increase in the emission of Cy3 (FIG. 22). Upon injection of ssCy3-DNA to a sample which contained protein (BSA and streptavidin) but no capture oligomers, there was no increase in Cy3 emission. The latter result shows that there was no significant non-specific binding of ssCy3-DNA to the protein surface which lacked the complementary oligomer. This result also shows there is little observable emission from ssCy3-DNA which was in the sample but not bound near the silver surface. In total, the data in FIG. 22 demonstrated that DNA hybridization can be detected from the SPCE. Furthermore, detection of hybridization depends on proximity to the silver surface and does not require a change in quantum yield of the fluorophore.

Background Suppression Using SPCE

The dependence of SPCE on proximity to the silver surface provides an advantage suppressing the background from regions of the sample more distal from the metal. The was tested by examining a sample containing both surface bound Cy3-DNA and non-complementary ssCy5-DNA. We measured both the SPCE and the free-space emission (FIG. 23). Free space emission refers to the non-directional emission which propagates away from the sample. For this measurement the probe was excited through the sample, and not through the prism, at normal incidence. In this configuration it is not possible to excite surface plasmons, so that the free space emission is similar to that which would be observed in a standard cuvette without a metal film.

At the excitation wavelength of 514 nm Cy5 absorbs light more weakly than Cy3. To obtain comparable intensities in the free space emission of Cy3 and Cy5 we used an approximate 30-fold higher concentration of ssCy5-DNA than Cy3-DNA, resulting in the free space emission spectrum shown in FIG. 23. We considered this comparable intensity due to Cy5 to be the unwanted background signal. We then changed the optical configuration to use surface plasmon (KR) excitation and to observe the SPCE. Using these conditions the emission was almost completely due to Cy3 (FIG. 23). The emission from Cy5 was suppressed 20-fold or more. Hence SPCE can be used with samples containing multiple fluorophores or autofluorescence, and only fluorophores close to the metal will result in SPCE.

SPCE can offer advantages for measurement of DNA hybridization and other binding interactions. When using the KR configuration excitation occurs selectively near the metal film due to the enhanced evanescent field. Irrespective of the use of the KR or RK configuration the increased intensity seen at the surface plasmon angle is due to fluorophore localization near the metal surface. Hence, binding can be detected without a change in probe intensity due to the binding event. Since the intensity change is due to surface localization, an intensity change can be observed for any association reaction and is not limited to fluorophores which display changes in quantum yield. Additionally, SPCE occurs over moderately large distances, typically up to several hundred nanometers from the metal surface. Since the biomolecules are typically much smaller, several layers of the capture molecules can be placed on the metal surface for increased sensitivity.

Another potential advantage of SPCE is effective rejection of the emission from fluorophores more distant from the metal. This suppression occurs by two mechanisms. One mechanism is decreased efficiency of coupling at larger distances from the metal. When using the Kretschmann configuration excitation occurs preferentially near the metal surface.

Another potential characteristic of SPCE is high sensitivity because plasmon coupling can result in light collection efficiency near 50%, much higher than efficiencies of a few percent with more typical optics. Note that SPCE can be observed with thin gold films which are chemically stable and for which the surface modification chemistry is well developed.

### Example 2

### Immunoassays

This example illustrates the use of SPCE in a model affinity assay. Goat anti-rabbit IgG antibodies against rabbit IgG bound to a 50 nm thick silver film were used. Binding of labeled IgG to the surface resulted in increased intensity observed at an angle of 75° from the normal in the glass substrate. The SPCE intensity depends on proximity of the fluorophore to the silver film, and does not require a change in quantum yield upon binding. The use of SPCE is shown to provide background suppression because excited fluorophores distant from the silver film do not result in SPCE. Sensitivity and selectivity can be further increased by excitation under conditions of surface plasmon resonance because the evanescent field is enhanced by the resonance interaction and excitation is limited to the region near the metal. SPCE can be used for high sensitivity and selectivity in surface-bound assays, the general principles demonstrated here can be applied to a wide variety of approaches, including, for example, microfluidic systems.

The usefulness of immunoassays depends on their sensitivity and specificity. Sensitivity is typically limited by the background auto-fluorescence which is present in all biological samples. Autofluorescence is also found in the optical elements of the instrumentation. In this example a model format for immunoassays using SPCE is illustrated, which provides increased sensitivity and background rejection by efficient light collection of emission occurring near the bioaffinity surface. The contribution of optical components to the background can preferably also be decreased due to an amplified excitation field, allowing the use of lower incident light intensities.

Materials and Methods

Glass microscope slides (Corning) were vapor deposited with a continuous 50 nm thick gold layer by EFM Company (Ithaca, N.Y.).

Rabbit IgG (anti-Mouse IgG produced in rabbit, total protein concentration 10 mg/mL, active antibody concentration 2.3 mg/mL) was from Sigma. Rhodamine Red-X— antiRabbit IgG (produced in goat) conjugate and AlexaFluor647-antiMouse IgG (produced in rabbit) conjugate (as stock solutions) were from Molecular Probes. Buffer components and salts (such as bovine serum albimun, glucose, sucrose, AgNO3) were from Sigma-Aldrich.

HPLC purified and concentrated bovine hemoglobin (HbBv) solution (˜17%) was used. Absorbance spectra taken at different dilutions of this HbBv solution showed that 1 mm thick layer of non-diluted HbBv solution has optical density (OD) of 5 at 514 nm, the excitation wavelength, and OD of 3 at 590 nm (the emission maximum of the bound labeled antibody).

Slides were non-covalently coated with rabbit IgG. 2 mL coating solution of IgG (30-50 mL of stock solution dissolved in 4 ml Na-phosphate buffer, 50 mM, pH 7.4) was added to the slide, and slide was incubated overnight at room temperature in a humid chamber. Slides were then rinsed with water, washing solution (0.05% Tween-20 in water), and water. Blocking was performed by adding 2.5 mL of blocking solution (1% BSA, 1% sucrose, 0.05% NaN3, 0.05% Tween-20 in 50 mM Tris-HCl buffer, pH 7.4) and incubation at 37° C. for 1 hour in humid chamber. The slide were rinsed with water, washing solution (0.05% Tween-20 in water), and water, covered with Na-phosphate buffer (50 mM, pH 7.4) and stored at +4° C. until use.

For an end-point binding experiment, dye-labeled conjugate Rhodamine Red-X-antiRabbit IgG (stock solution diluted 200 times with Na-phosphate buffer, 50 mM, pH 7.4) was added to the slide (coated with rabbit IgG as described above) and incubated at 37° C. in a humid chamber for 1.5 hours. Slide then was rinsed with water, washing solution (0.05% Tween-20 in water), and water. Then, a rubber ring (7 mm diameter and 9 mm height) was placed on the metallic side of the slide and covered with a second glass slide. About 1.5 ml of the Na-phosphate buffer, 50 mM, pH 7.4, was added inside the rubber ring chamber using needle, and fluorescence measurements were performed at two different optical configurations (Kretschmann and reverse Kretschmann).

To test the effect of a background two different solutions were added inside the rubber ring cell; 1) a highly absorbing HbBv solution as non-fluorescent background or 2) a highly fluorescent solution of non-binding AlexaFluor647-antiMouse IgG conjugate. The HbBv solution was used undiluted and AlexaFluor647-antiMouse IgG conjugate was used at 500-fold dilution.

For a kinetic binding experiment, a rubber ring (7 mm diameter and 9 mm height) was placed on the surface of the metal mirror slide (coated with Rabbit IgG as described above) and covered with a second glass slide. About 1.5 ml of the dye-labeled conjugate Rhodamine Red-X-antiRabbit IgG (stock solution diluted 200 times with Na-phosphate buffer, 50 mM, pH 7.4) was added inside the rubber ring chamber using needle. Kinetics was immediately monitored at room temperature.

Spectroscopic Measurements

Absorption spectra were measured on Hewlett Packard model 8543 spectrophotometer using 1 cm cuvettes. Emission measurements in cuvettes were performed using a Varian Eclipse spectroflurometer. For fluorescence measurements with microscope slides used index-matching fluid to a hemicylindrical prism made of BK7 glass and positioned on a precise rotatory stage equipped with the fiber optics mount on a 15 cm long arm. This configuration allowed fluorescence observation at any angle relative to the incident angle. The outlet of the fiber was connected to an Ocean Optics SD2000 spectrofluormeter for emission spectra and intensity measurements. The excitation was from a pulsed mode-locked argon ion laser (Coherent). Scattered and reflected incident light (514 nm) was suppressed on observation by using a holographic supernotch-plus filter (Kaiser Optical System, Ann Arbor, Mich.).

Results

The device used for this model SPCE immunoassay is shown in FIG. 25. The sample was held in a cylindrical volume by an o-ring between two slides, the upper slide being coated with a 50 nm silver film. Rhodamine-labeled IgG was bound near the gold surface by its binding to the surface-bound antigen. Initially the sample was illuminated in the RK configuration, which does not create surface plasmons in response to the incident light. We measured the emission intensity for all accessible angles from the normal axis (FIG. 26). The intensity observed through the prism was sharply directed near ±75°. This value is in good agreement with that calculated from minimum reflectance for p-polarized plasmon mode.

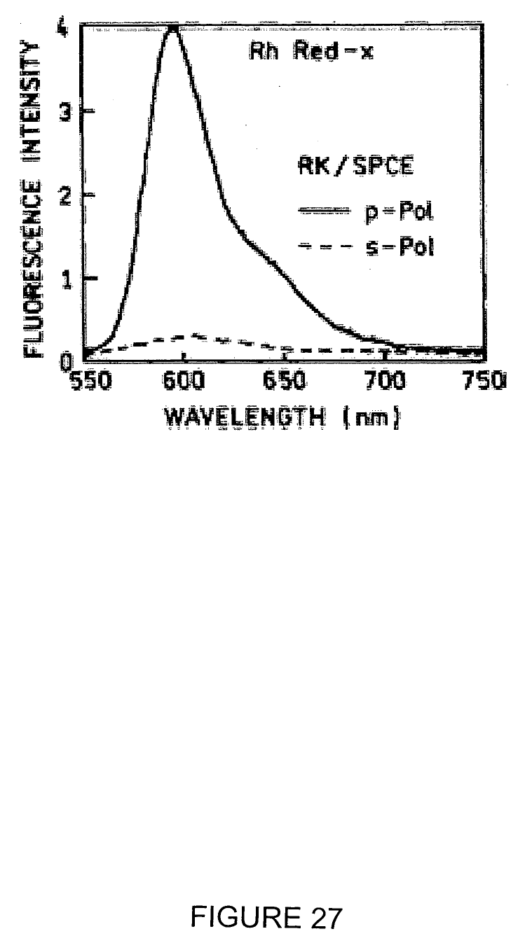

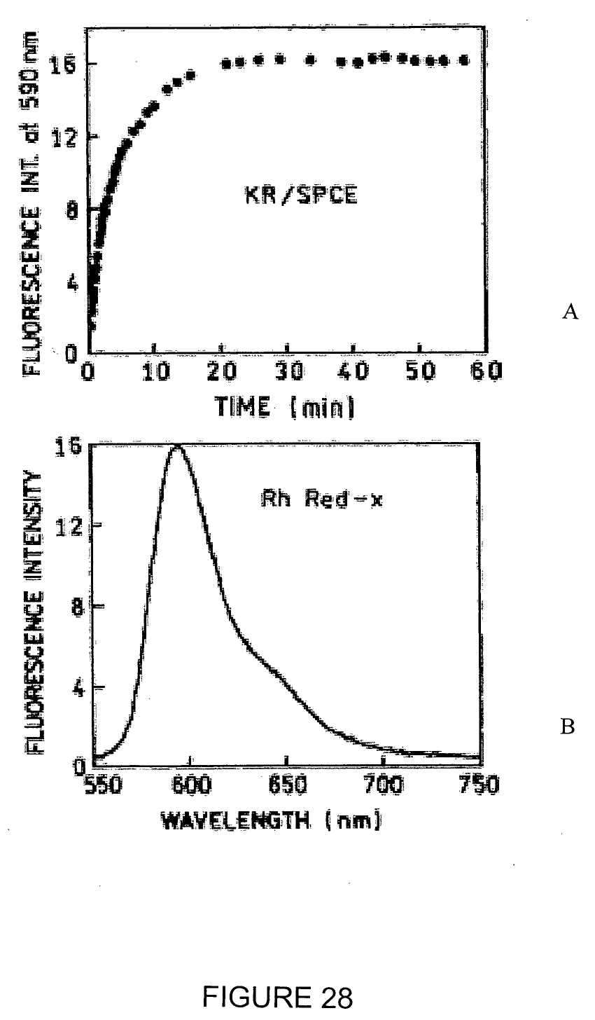

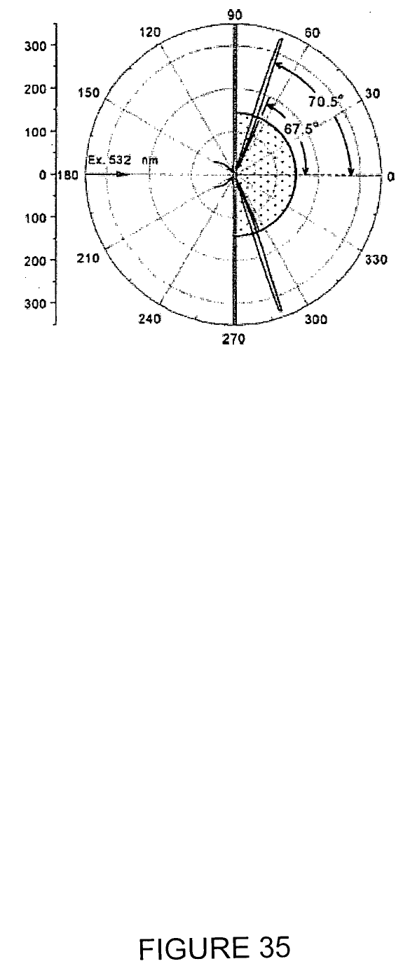

The emission spectrum of the SPCE was characteristic of the rhodamine probe (FIG. 35) and spectrum was not corrupted by scattered light at the excitation wavelength. An unusual characteristic of SPCE is the near complete polarization in the p-direction, meaning the electric vector is oriented parallel to the plane of incidence. FIG. 27 shows the emission spectra collected through an emission polarizer oriented p or s. The orientation of the excitation polarizer did not affect these intensities. The p-polarized intensity is 20-fold more intense, resulting in a polarization of p≈0.9. This large value cannot be the result of photoselection in an isotropic media. Also, this value is independent of the orientation of the excitation polarization. This p-polarization proves that the emission is due to surface plasmons, which under these conditions cannot emit s-polarized light.

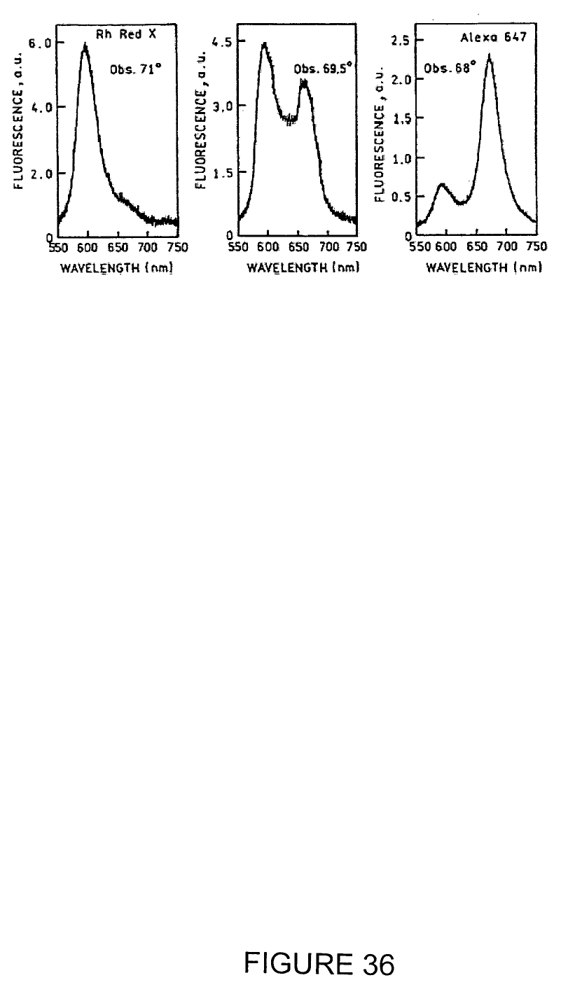

We tested the use of SPCE to measure the binding kinetics of the rhodamine-labeled antibodies to the surface bound antigen. FIG. 36 shows the emission intensities after adding labeled antibody. The emission climbs rapidly and reaches a limiting value. This 10-fold change in intensity is not the result of a change in the rhodamine quantum yield upon binding. We measured the effect of binding the rhodamine-labeled goat antibody to the antigen while both were free in solution, and found the intensity decrease due to binding was about 25%. This indicates the intensity change is due to localization of the probe in the evanescent field near the gold. Thus the use of SPCE is a generic method to detection of surface localization by a change in intensity, but does not require a change in the fluorophore quantum yield upon binding.

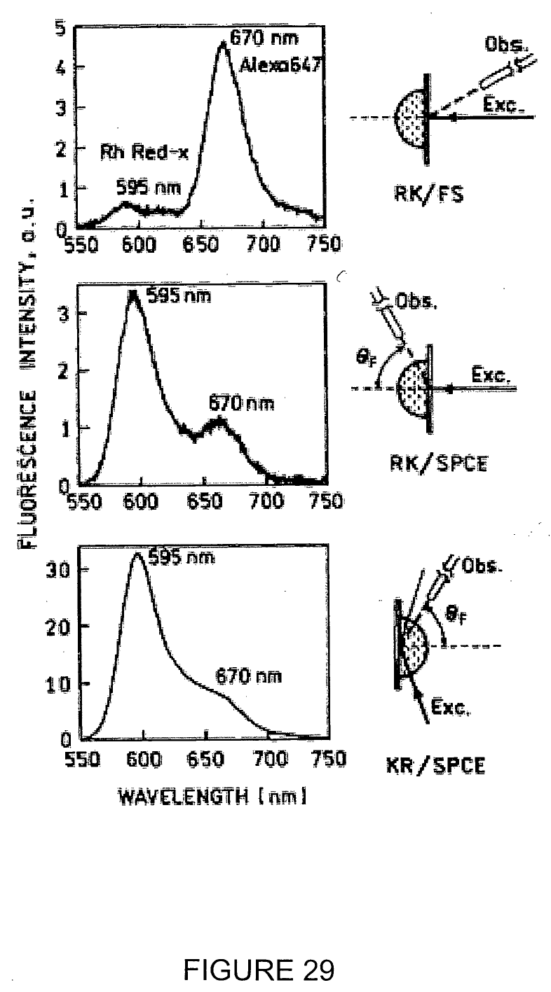

We tested several optical configurations to determine the relative intensities and extent of background rejection possible using SPCE. These three configurations are shown in the right-hand panels in FIG. 29. This sample consisted of the surface saturated with rhodamine-labeled antibody. We then added Alexa 647-labeled antibody (not binding to the surface) to mimic autofluorescence from the sample. The 15 μM concentration of this antibody (65 μM of Alexa dye) resulted in dominant free-space fluorescence signal from the sample. First the sample was excited using the RK configuration, and the free space emission observed from the same water side of the sample. Compared to subsequent measurements the intensity of the desired rhodamine antibody (below) the signals were weak. The free-space emission was dominated by the emission from Alexa at 670 nm, with only weak rhodamine emission at 595 nm. We then measured the emission spectrum of the SPCE signal (middle panel), while still using RK illumination. The emission spectrum was dramatically changed from a 10-to-1 excess of the unwanted background to a 5-to-1-excess of the desired signal. Hence, the use of SPCE resulted in selective detection of the rhodamine-labeled antibody near the silver film.

The mode of excitation was then changed to the KR configuration (FIG. 29, bottom panel). In this case the sample was illuminated at θ_{SP} creating an evanescent field in the sample. The overall intensity was increased 10-fold while further suppressing the unwanted emission from Alexa 647. The increased intensity and decreased background is the result of localized excitation by the resonance-enhanced field near the metal. In this case the emission was due almost entirely to the rhodamine, with just a minor contribution from the Alexa-labeled protein.

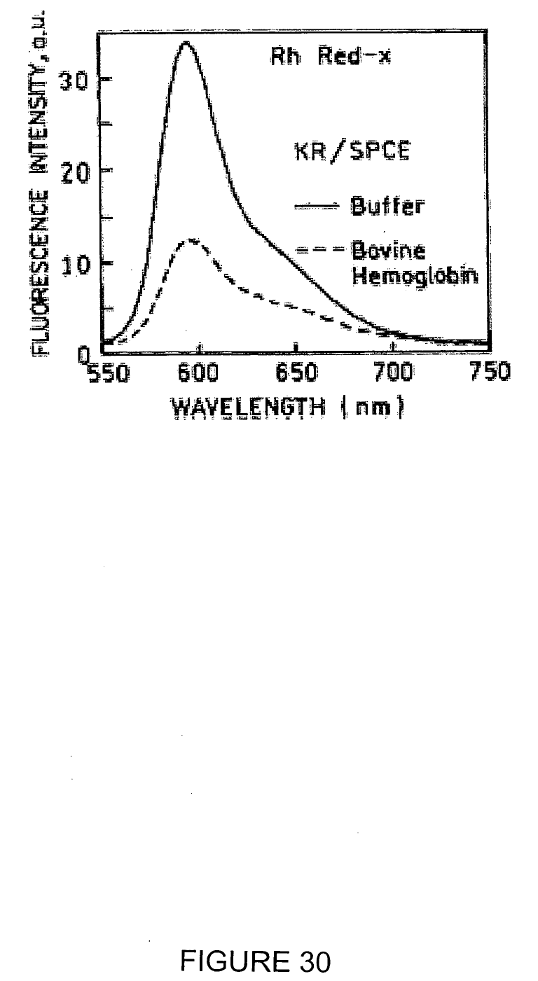

In medical testing it is often desirable to perform homogenous assays without separation steps, sometimes in whole blood. It appears that SPCE should be detectable in optically dense media because this signal arises from the sample within 200 nm of the surface. To mimic whole blood we added 17% bovine hemoglobin solution, which had an optical densities of 5 and 3 at 514 and 590 nm, respectively. In a 1.0 mm thick sample these optical densities would attenuate the signal about a million-fold. Using SPCE the signal was attenuated less than 3-fold (FIG. 30). These results show the potential of using SPCE in optically dense samples.

### Example 3

### Multi-Wavelength Immunoassays

This example illustrates a method for multi-wavelength immunoassays using surface plasmon-coupled emission, SPCE, The angle at which emitted radiation propagates through the prism depends on the surface plasmon angle for the relevant wavelength. These angles depend on emission wavelength, allowing measurement of multiple analytes using multiple emission wavelengths. We demonstrated this using antibodies labeled with either Rhodamine Red-X or AlexaFluor 647. These antibodies were directed against an antigen protein bound to the silver surface. The emission from each labeled antibody occurred at a different angle on the glass prism, allowing independent measurement of surface binding of each antibody. This method of SPCE immunoassays can be readily extended to 4 or more wavelengths.

Materials and Methods

Rabbit IgG (11.2 mg/ml) was from Sigma. Rhodamine Red-X-antiRabbit IgG (produced in goat, 2 mg/mL, dye/protein =3.8 mol/mol) and AlexaFluor647-antiRabbit IgG (produced in goat, 2 mg/mL, dye/protein=4.5 mol/mol) conjugates were from Molecular Probes. Buffer components and salts (such as bovine serum albumin, glucose, sucrose) were from Sigma-Aldrich.

Standard glass microscope slides (3×1 inch, 1 mm thick; Corning) were vapor deposited with a continuous 50 nm thick silver layer by EMF Corp. (Ithaca, N.Y.). Slides were non-covalently coated with Rabbit IgG: 1.6 ml coating solution of IgG (50 μg/mL IgG in Na-phosphate buffer, 50 mM, pH 7.4) was added to the slide, and slide was incubated 3.5 hours at room temperature in a humid chamber. Slides were then rinsed with water, washing solution (0.05% Tween-20 in water), and water. Blocking was performed by adding 2.0 mL of blocking buffer (1% bovine serum albumin (BSA), 1% sucrose, 0.05% NaN3, 0.05% Tween-20 in 50 mM Tris-HCl buffer, pH 7.4) and incubation overnight at +4° C. in humid chamber. The slides were rinsed with water, washing solution (0.05% Tween-20 in water), and water, covered with blocking buffer and stored at +4° C. until use.

End-Point Binding Experiment

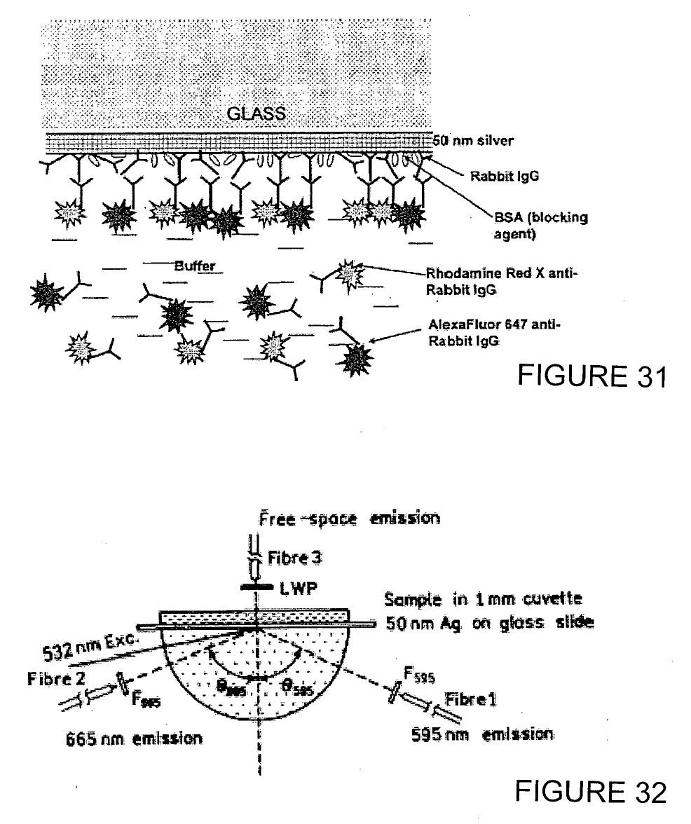

Two dye-labeled conjugates, Rhodamine Red-X-antiRabbit IgG (RhX-Ab) and AlexaFluor647-antiRabbit IgG (Alexa-Ab) were mixed in blocking solution: 20 μL of each stock conjugate solution was added to 4 mL of blocking solution. This mixture was added to the slides (1.5 mL per slide), and slides were incubated at 37° C. in a humid chamber for 1 hour. Slides then were rinsed with water, washing solution (0.05% Tween-20 in water) and water. A 1 mm thick cuvette was mounted on the metallic side of the slide. About 0.4 ml of the blocking buffer was added inside the cuvette, and fluorescence measurements were performed at two different optical configurations (Kretschmann and reverse Kretschmann). See FIG. 31.

Kinetic Binding Experiment

A 1 mm thick cuvette was mounted on the metallic side of the slide (coated with Rabbit IgG as described above). About 0.4 ml of the mixture of two dye-labeled conjugates Rhodamine Red-X-antiRabbit IgG and AlexaFluor647-antiRabbit IgG (prepared as described above) was added into the cuvette using a needle. Kinetics was immediately monitored at room temperature (17° C.) using the configuration shown in FIG. 32.

Spectroscopic Measurements

Fluorescence measurements on microscope slides were performed using index-matching fluid to attach the slide to a hemicylindrical prism made of BK7 glass and positioned on a precise rotatory stage equipped with the fiber optics mount on a 15 cm long arm. This configuration allowed fluorescence observation at any angle relative to the incident angle. The output of the fiber was connected to an Ocean Optics SD2000 spectrofluorometer for emission spectra. The excitation at 532 nm was from a solid-state laser (maximal output power 30 mW). The kinetic measurements were done with simultaneous observation through three fibers pointing to three independent detectors.

The experimental configuration used for two-wavelength SPCE is shown in FIG. 32. The protein-coated silver surface is illuminated at the surface plasmon angle through the glass prism, which is called the Kretschmann (KR) configuration. The free-space emission is observed normal to the sample surface, on the side distal from the prism, using a filter and a fiber optic bundle. SPCE is observed on the prism side of the sample, at two different angles through appropriate long-pass filters for each labeled antibody. The sample can also be excited through the aqueous phase in the reverse Kretschmann (RK) configuration.

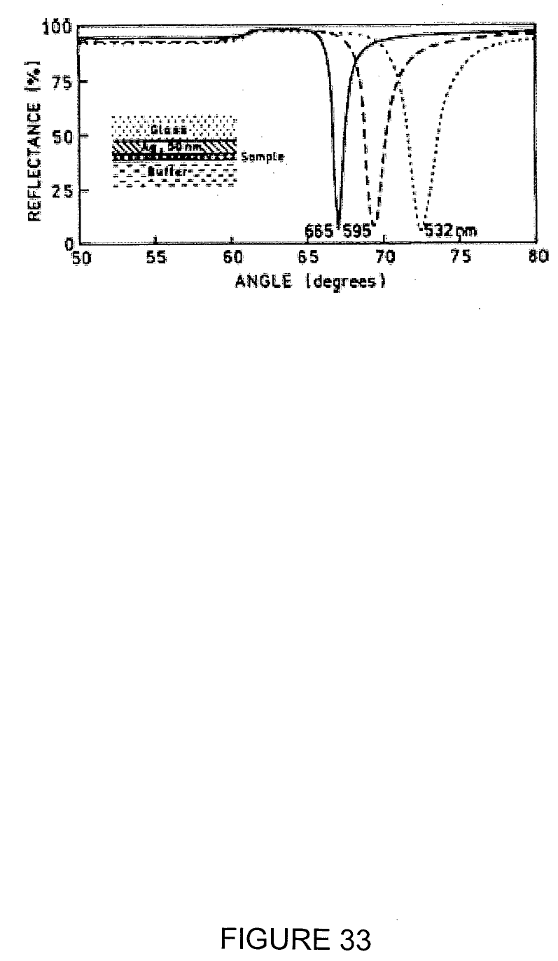

As described above, there can be close agreement between the calculated reflectivity minima for the emission wavelength and the observed SPCE angles. Hence we calculated the reflectivity expected for over the 50 nm thick silver films and our optical configuration (FIG. 33). The reflectivity curves can be calculated using either software available on the web or commercial software which we found to yield equivalent results. The reflectivity minima were found at 72.5° for 532 nm, and at 69° and 67° for 595 and 665 nm, respectively. Hence we expected to obtain excitation of surface plasmons with a 532 nm incident angle of 72.5°, and to observe the RhX-Ab and Alexa-Ab emission at 69° and 67°, respectively.

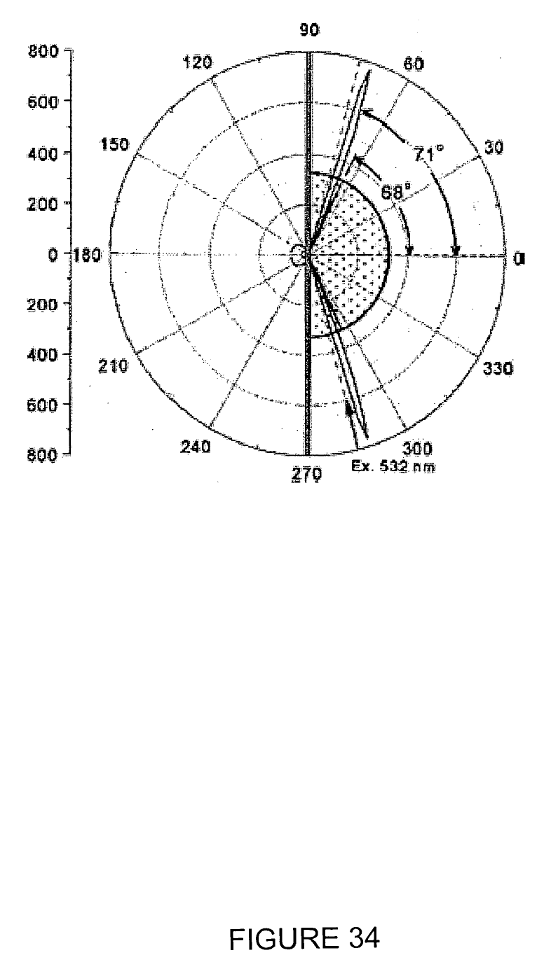

We examined the angle-dependent emission intensity for an antigen (rabbit IgG) covered surface which was saturated with a mixture of RhX-Ab and Alexa-Ab (FIG. 34). The emission from both labeled antibodies was strongly directional at different angles on the prism. The emission from RhX-Ab peaked at 71°, and that from Alexa-Ab at 68°. Preferably, SPCE does not require excitation of surface plasmons by the incident light. To demonstrate this fact we excited the sample through the aqueous phase (FIG. 35). Once again the emission from each labeled antibody was strongly directional in the prism at the surface plasmon angle for the emission wavelength. This result illustrates that SPCE occurs via an interaction of the excited fluorophores with the metal surface and does not depend on creation of surface plasmons by the incident light.

The angle-dependent intensities in FIGS. 42 and 43 were collected through a emission filter to isolate the emission from each labeled antibody. However, these measurements did not resolve the emission spectra of each antibody. FIG. 4 shows emission spectra collected using observation angles of 71°, 69.5° and 68°. At 71° the emission is almost completely due to RhX-Ab with an emission maximum of 595 nm. At 68° the emission is due mostly to Alexa-Ab at 665 nm, with a residual component from RhX-Ab at 595 nm. At the intermediate angle of 69.5° the emission from both labeled antibodies is seen. These emission spectra show that the desired emission wavelength can be selected by adjustment of the observation angle.

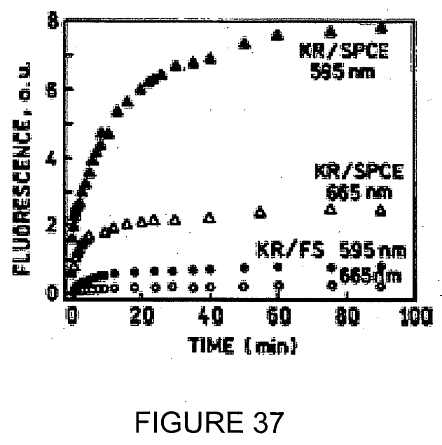

We used SPCE at two observed angles to simultaneously measure the binding kinetics of both labeled antibodies (FIG. 37). The binding kinetics were similar even through the final intensities are different. The binding was also measured using KR excitation and the free-space emission. The intensities are over 10-fold higher for SPCE than for the free-space emission.

The silver film can preferably serve multiple purposes. For example, it can amplify the incident light, efficiently collect the emission, and provide separation of the wavelengths. Detection could be accomplished with imaging or point detectors, to provide a simple yet sensitive device. The number of analytes can be increased by using fluorophores with emission wavelengths ranging from 450 to 800 nm. Still more analytes could be measured using semiconductor nanoparticles, which display narrow emission spectra. As described above, the angular dependence on wavelength can be increased using thin (50 nm) or thick (200 nm) metal gratings, which also display SPR with an additional dependence on the grating constant. The use of the KR configuration localizes the excitation near the metal surface, but further localization is possible using multi-photon excitation. The surface chemistry of silver and gold is well developed, and we have observed SPCE using gold films.

1. An apparatus for detecting fluorescence in biochemical assays using surface plasmon-coupled emission, comprising:
a first layer of conductive material arranged on a first medium, the first medium having a first index of refraction and being a solid medium, said first layer of conductive material being situated at an interface between said first medium and a second medium, the second medium having a second index of refraction different from the first index of refraction;
a second layer comprising functional molecules disposed on the first layer, the functional molecules comprising at least one of nucleic acid molecules and polypeptide molecules, the functional molecules comprising one or more types of fluorophores and/or being capable of binding analyte molecules comprising one or more types of fluorophores;
an excitation source capable of exciting fluorophores positioned adjacent to the first layer; and
a light detector arranged to selectively detect emitted light that is generated by excited fluorophores, the detector being arranged to collect emitted light over a predetermined angular range relative to a surface of the first medium, said emitted light emanating from the first layer at the surface plasmon angle for an emission wavelength of the excited fluorophores relative to a surface of said first layer and passing through the first medium before being detected by the detector, the predetermined angular range comprising the surface plasmon angle for the emission wavelength of the excited fluorophores.

2. The apparatus of claim 1, wherein the excitation source comprises a light source capable of producing light comprising an excitation wavelength of fluorophores, the light source being arranged to direct light from the light source toward the first layer.

3. The apparatus of claim 1, further comprising a third layer arranged between the first layer and the second layer, the third layer comprising at least one of silica, polymer material, protein molecules or lipid molecules.

4. The apparatus of claim 1, wherein the first layer comprises a metal.

5. The apparatus of claim 1, wherein the metal is deposited onto the first medium by vapor deposition, electroless plating, chemical vapor deposition, or photoreduction.

6. The apparatus of claim 1, wherein the first layer comprises silver, gold, aluminum, or copper.

7. The apparatus of claim 1, wherein the first medium comprises a glass plate, a silica substrate, a polymer substrate, or a prism.

8. The apparatus of claim 1, wherein the apparatus comprises a glass prism, a glass plate coated with the first layer of conductive material on a side of the plate facing away from the prism, and an index matching fluid having substantially the same index of refraction as the glass prism and the glass plate, the index matching fluid being disposed between the glass prism and glass plate.

9. The apparatus of claim 1, wherein the second medium comprises an aqueous solution, a polymer, or air.

10. The apparatus of claim 2, wherein said light source is arranged to direct light comprising said excitation wavelength through said first medium and then to said first layer such that the angle of incidence on the first layer is equal to the surface plasmon angle of said excitation wavelength.

11. The apparatus of claim 2, wherein the second layer is configured to position fluorophores within an evanescent field at the first layer, the evanescent field being generated by light from the light source.

12. The apparatus of claim 2, wherein said light source is arranged to direct light comprising said excitation wavelength through said second medium and then to said first layer.

13. The apparatus of claim 1, comprising a focusing element that receives a hollow cone of light emitted by the flourophores and that focuses a portion of the hollow cone of light onto a detector, wherein said detector is arranged to selectively detect the focused light.

14. The apparatus of claim 13, wherein said focusing element comprises a lens situated between said first medium and said detector.

15. The apparatus of claim 13, wherein said focusing element comprises a prism that is capable of redirecting light emitted at the thin layer of conductive material by total internal reflection.

16. The apparatus of claim 15, wherein said prism forms all or part of said first medium.

17. The apparatus of claim 15, wherein said prism has a shape selected from among polygonal, hemispherical, and spherical shapes.

18. The apparatus of claim 1, wherein the first layer comprises a patterned structure.

19. The apparatus of claim 18, wherein the first layer comprises a plurality of apertures arranged therein.

20. The apparatus of claim 19, wherein the plurality of apertures have a substantially uniform size and are arranged in a predetermined pattern.

21. The apparatus of claim 18, wherein the first layer is discontinuous and comprises a plurality of ring-shaped regions of conductive material arranged in a predetermined pattern on the first medium.

22. The apparatus of claim 18, wherein the first layer comprises a grating structure.

23. The apparatus of claim 1, wherein the second layer comprises a plurality of functional regions, the functional regions being separated from one another laterally and being arranged in a predetermined pattern on the first layer, at least some of the plurality of functional regions comprising functional molecules that are different from functional molecules of other ones of the plurality of functional regions.

24. The apparatus of claim 23, wherein the detector selectively detects light from individual functional regions.

25. The apparatus of claim 2, further comprising a mechanism that allows the light source to successively illuminate different positions on the first layer and that allows the detector to detect the light generated by the fluorophores as a function of the illumination of the different positions, the detector thereby generating two-dimensional data from the light generated by the fluorophores.

26. The apparatus of claim 25, wherein said mechanism is a translator that provides relative motion between the first medium and the light source and between the first medium and the detector.

27. The apparatus of claim 1, wherein the second layer is configured to position the fluorophores within about 5 to 500 nm of the first layer.

28. The apparatus of claim 2, wherein the light source is configured to illuminate a selected region of the second layer, the apparatus further comprising a time-domain recorder coupled to said detector to thereby record a signal from said detector as a function of time, said signal corresponding to light generated by fluorophores at said selected region.

29. The apparatus of claim 1, wherein:
the functional molecules either comprise a plurality of types of fluorophores or are bound to analyte molecules comprising a plurality of types of fluorophores;
fluorescence emission of each type of fluorophore has a different emission wavelength; and
the detector is configured to selectively detect light generated by each type of fluorophore by collecting light generated by different types of fluorophores at different angles.

30. The apparatus of claim 29, wherein the first layer comprises a patterned structure that provides further angular separation between light generated by different types of fluorophores.

31. The apparatus of claim 1, further comprising conductive particles having diameters less than about 200 nm disposed on the first layer.

32. A method for detecting fluorescence in biochemical assays using surface plasmon-coupled emission, comprising:
arranging an assay device proximate to a light detector, the assay device comprising a first layer of conductive material arranged on a first medium, the first medium having a first index of refraction and being a solid medium, said first layer of conductive material being situated at an interface between said first medium and a second medium, the second medium having a second index of refraction different from the first index of refraction, the assay device further comprising a second layer comprising functional molecules disposed on the first layer, the functional molecules comprising at least one of nucleic acid molecules and polypeptide molecules, the functional molecules being capable of binding analyte molecules comprising one or more types of fluorophores;
causing fluorophores to be adjacent to said first layer of said assay device;
exciting at least some of said fluorophores with an excitation source; and
detecting emitted light that is generated by excited fluorophores with a detector, said emitted light having an emission wavelength of the fluorophores, said emitted light emanating from said first layer of conductive material at the surface plasmon angle of said emission wavelength relative to a surface of said first layer and passing through said first medium before being detected by the detector.

33. The method of claim 32, wherein the excitation source comprises a light source capable of producing light comprising an excitation wavelength of the fluorophores adjacent to the first layer of said assay device, and wherein exciting at least some of said fluorophores comprises illuminating at least some of said fluorophores with light from the light source.

34. The method of claim 32, wherein causing said fluorophores to be adjacent to said first layer comprises causing said fluorophores to be within about 5-500 m of said first layer.

35. The method of claim 32, wherein causing said fluorophores to be adjacent to said first layer comprises applying a coating comprising said fluorophores onto said first layer.

36. The method of claim 32, wherein causing said fluorophores to be adjacent to said first layer comprises:
exposing said second layer that comprises said functional molecules to analyte molecules that comprise said fluorophores; and
allowing said analyte molecules to bind to said functional molecules.

37. The method of claim 36, wherein the second layer comprises a plurality of functional regions, the functional regions being separated from one another laterally and being arranged in a predetermined pattern on the first layer, at least some of the plurality of functional regions comprising functional molecules that are different from functional molecules of other ones of the plurality of functional regions, the method further comprising selectively detecting light from individual functional regions with said detector.

38. The method of claim 36, wherein exposing said second layer comprises exposing said second layer to said first medium, the first medium being a fluid medium.

39. The method of claim 36, comprising exposing said second layer to a plurality of substances comprising a plurality of different types of fluorophores.

40. The method of claim 32, wherein said analyte molecules comprise at least one of antibodies, fragments of an antibodies, peptide antigens, nucleic acids, and polypeptides, and wherein said analyte molecules comprise one or more types of fluorophores.

41. The method of claim 39, wherein fluorescence emission of each type of fluorophore has a different emission wavelength, and wherein emission from each type of fluorophore is selectively detected by collecting light emitted at an angle corresponding to the surface plasmon angle for the emission wavelength of each type of fluorophore.

42. The method of claim 33, wherein illuminating at least some of said fluorophores comprises illuminating said second layer through the second medium.

43. The method of claim 33, wherein illuminating at least some of said fluorophores comprises illuminating said first layer through the first medium, and wherein said light is directed at said first layer at a surface plasmon resonance angle of the excitation wavelength.

44. The method of claim 32, wherein detecting emitted light comprises selectively detecting light emitted into said first medium in the form of a hollow cone and that has been directed to said detector by a focusing element.

45. The method of claim 44, wherein said focusing element comprises a lens situated between said first medium and said detector.

46. The method of claim 44, wherein said focusing element comprises a prism that is capable of redirecting light emitted at the thin layer of conductive material by total internal reflection.

47. The method of claim 46, wherein said prism forms all or part of said first medium.

48. The method of claim 46, wherein said prism has a shape selected from among polygonal, hemispherical, and spherical shapes.

49. The method of claim 32, further comprising passing the emitted light through a third layer arranged between the first layer and the second layer before detecting the emitted light with the detector, the third layer comprising at least one of silica, polymer material, protein molecules or lipid molecules.

50. The method of claim 32, wherein the first layer comprises a metal.

51. The method of claim 32, wherein the metal is deposited onto the first medium by vapor deposition, electroless plating, chemical vapor deposition, or photoreduction.

52. The method of claim 32, wherein the first layer comprises silver, gold, aluminum, or copper.

53. The method of claim 32, wherein the first medium comprises a glass plate, a silica substrate, a polymer substrate, or a prism.

54. The method of claim 32, comprising passing the emitted light from the first medium through an index matching fluid and through a glass prism before detecting the emitted light with a detector, the first medium comprising a glass plate coated with the thin layer of conductive material on a side of the plate facing away from the prism, the index matching fluid having substantially a same index of refraction as the glass prism and the glass plate, the index matching fluid being disposed between the glass prism and glass plate.

55. The method of claim 32, wherein the second medium comprises an aqueous solution, a polymer, or air.

56. The method of claim 33, comprising positioning the fluorophores within an evanescent field at the first layer, the evanescent field being generated by light from the light source.

57. The method of claim 32, wherein the first layer comprises a patterned structure.

58. The method of claim 57, wherein the first layer comprises a plurality of apertures arranged therein.

59. The method of claim 58, wherein the apertures have a substantially uniform size and are arranged in a predetermined pattern.

60. The method of claim 57, wherein the first layer is discontinuous and comprises a plurality of ring-shaped regions of conductive material arranged in a predetermined pattern on the first medium.

61. The method of claim 57, wherein the first layer comprises a grating structure.

62. The method of claim 33, further comprising successively illuminating different positions on the first layer and detecting the light generated by the fluorophores as a function of the illumination of the different positions, the detector thereby generating two-dimensional data from the light generated by the fluorophores.

63. The method of claim 33, comprising illuminating a selected region of the second layer, and recording a signal generated by the detector as function of time with a time-domain recorder coupled to said detector, said signal corresponding to light generated by fluorophores at said selected region.

64. An apparatus for observing surface plasmon-coupled emission, comprising:
an optical fiber having a first index of refraction and having a surface portion coated with a first layer of conductive material, the first layer of conductive material being situated at an interface between the optical fiber and a medium, the medium having a second index of refraction different from the first index of refraction;
a second layer comprising functional molecules disposed on the first layer, the functional molecules comprising at least one of nucleic acid molecules and polypeptide molecules, the functional molecules comprising one or more types of fluorophores and/or being capable of binding to analyte molecules comprising one or more types of fluorophores;
an excitation source capable of exciting fluorophores positioned adjacent to the first layer; and
a light detector optically coupled to the optical fiber and arranged to collect emitted light generated by excited fluorophores, said emitted light passing through the optical fiber to the detector, the emitted light having an emission wavelength of the fluorophores.

65. An method for observing surface plasmon-coupled emission, comprising:
optically coupling an optical fiber to a light detector, the optical fiber having a first index of refraction and having a surface portion coated with a first layer of conductive material, the first layer of conductive material being situated at an interface between the optical fiber and a medium, the medium having a second index of refraction different from the first index of refraction, the optical fiber further having a second layer comprising functional molecules disposed on the first layer, the functional molecules comprising at least one of nucleic acid molecules and polypeptide molecules, the functional molecules comprising one or more types of fluorophores and/or being capable of binding to analyte molecules comprising one or more types of fluorophores;
causing fluorophores to be adjacent to said first layer of conductive material;
exciting at least some of said fluorophores adjacent to said first layer with an excitation source; and
detecting light generated by excited fluorophores with the detector, the emitted light passing through the optical fiber to the detector, the emitted light having an emission wavelength of the fluorophores.

66. An apparatus for observing surface plasmon-coupled emission, comprising:
a layer of conductive material arranged on a first medium, the first medium having a first index of refraction and being a solid medium, the layer of conductive material being situated at an interface between the first medium and a second medium, the second medium having a second index of refraction different from the first index of refraction, the layer of conductive material comprising a patterned structure;
one or more types of fluorophores positioned adjacent to said layer of conductive material;
an excitation source capable of exciting fluorophores positioned adjacent to the layer of conductive matieral; and
a light detector arranged to selectively detect emitted light that is generated by excited fluorophores, the detector being arranged to collect emitted light over a predetermined angular range relative to a surface of the first medium, said emitted light emanating from the layer of conductive material at the surface plasmon angle for an emission wavelength of the excited fluorophores relative to a surface of the layer of conductive material and passing through the first medium before being detected by the detector, the predetermined angular range comprising the surface plasmon angle for the emission wavelength of the excited fluorophores.

67. An method for observing surface plasmon-coupled emission, comprising:
arranging a first medium proximate to a light detector, the first medium having a layer of conductive material arranged on a surface thereof, the first medium having a first index of refraction and being a solid medium, said layer of conductive material being situated at an interface between said first medium and a second medium, the second medium having a second index of refraction different from the first index of refraction, the layer of conductive material comprising a patterned structure;
causing one or more types of fluorophores to be adjacent to said layer of conductive material;
exciting at least some of said fluorophores with an excitation source; and
detecting emitted light that is generated by excited fluorophores with a detector, said emitted light having an emission wavelength of the fluorophores, said emitted light emanating from said layer of conductive material at the surface plasmon angle of said emission wavelength relative to a surface of said layer of conductive material and passing through said first medium before being detected by the detector.

68. A method of imaging fluorescence emission from one or more types of fluorophores bound to cellular sample, comprising:
placing a cellular sample on a layer of conductive material disposed on a first medium, the first medium having a first index of refraction and being a solid medium, said layer of conductive material being situated at an interface between said first medium and a second medium, the second medium having a second index of refraction different from the first index of refraction;
exposing said cellular sample to one or more substances capable of binding to one or more types of molecules in said cellular sample, said substances comprising one or more types of fluorophores, thereby causing fluorophores to be adjacent to said layer of conductive material;
illuminating a selected position on said layer of conductive material at an excitation wavelength of said fluorophores;
detecting emitted light that is generated by excited fluorophores at the selected position with a detector, said emitted light having an emission wavelength of the fluorophores, said emitted light emanating from said layer of conductive material at the surface plasmon angle of said emission wavelength relative to a surface of said layer of conductive material and passing through said first medium before being detected by the detector; and
successively illuminating new selected positions on said layer of conductive material and detecting light emitted at each new selected position.

69. The method of claim 68, wherein the cellular sample is a tissue sample.

70. The apparatus of claim 1, wherein an intensity of the emitted light at said surface plasmon angle from said fluorophores adjacent to the first layer of conductive material is enhanced relative to emission from fluorophores located distant from said first layer of conductive material, thereby effectively suppressing detection of background emission relative to detection of the emitted light from said fluorophores adjacent to the first layer of conductive material.

71. The method of claim 32, wherein an intensity of the emitted light at said surface plasmon angle from said fluorophores adjacent to the first layer of conductive material is enhanced relative to emission from fluorophores located distant from said first layer of conductive material, thereby effectively suppressing detection of background emission relative to detection of the emitted light from said fluorophores adjacent to the first layer of conductive material.
